(19) United States
(12) Patent Application Publication
(10) Pub. No.: US 2025/0085264 A1
(43) Pub. Date: Mar. 13, 2025

(54) METHOD AND DEVICE FOR IMPROVING THE QUALITY AND TRACEABILITY OF ALCOHOLIC BEVERAGES, IN PARTICULAR WINES

(71) Applicants: M&WINE, Fontaines Saint-Martin (FR); CENTRE NATIONAL DE LA RECHERCHE SCIENTIFIQUE, Paris (FR); UNIVERSITE CLAUDE BERNARD LYON 1, Villeurbanne Cedex (FR); INSTITUT NATIONAL DES SCIENCES APPLIQUEES DE LYON, Villeurbanne Cedex (FR); UNIVERSITE JEAN MONNET SAINT-ETIENNE, Saint Etienne Cedex 2 (FR)

(72) Inventors: Olivier TILLEMENT, Fontaines Saint-Martin (FR); Théodore TILLEMENT, Fontaines Saint-Martin (FR); François LUX, Lyon (FR); Fabien ROSSETTI, Villeurbanne (FR); Matteo MARTINI, Villars les Dombes (FR); Pierre DER NIGOHOSSIAN, Collonges au Mont d'Or (FR); Agnes HAGEGE, Lyon (FR); Laurent DAVID, Lyon (FR)

(21) Appl. No.: 18/721,995

(22) PCT Filed: Dec. 27, 2022
(86) PCT No.: PCT/FR2022/052512

§ 371 Date: Jun. 20, 2024

(30) Foreign Application Priority Data
Dec. 29, 2021 (FR) 2114652

(51) Int. Cl. G01N 33/14; C12G 1/00; G06Q 30/018

(52) U.S. Cl. CPC G01N 33/146; C12G 1/00; G06Q 30/0185

(57) ABSTRACT

A computer-implemented method for managing/monitoring production of agricultural raw materials useful for the production of alcoholic beverages, as well as the production, storage, ageing, consumption, quality, authenticity, traceability and/or selling price of alcoholic beverages comprises: (a) collecting two samples of the alcoholic beverage in an inert container sealed with an inert stopper; (b) assigning data on the alcoholic beverage to each sample; (c) storing at least some of the samples collected in step (a), under specified conditions; (d) analysing each sample to determine at least one mineral profile, preferably metallic; (e) forming a database relating to the samples and resulting from step (b) and step (d); (h) processing these data by statistical analysis, preferably using AI; and (i) using the processed data to manage/monitor the entire supply chain of the alcoholic beverage until its consumption. A device for implementing steps (a) and (c) of the method is also provided.

### FIELD OF THE INVENTION

The invention relates to the field of preparing alcoholic beverages, in particular wines, from a raw material derived from agriculture, in this case from viticulture for wines.

More specifically, the invention relates to a type of technology that aims to improve the quality and the traceability of alcoholic beverages, in particular wines (with the term “wine” hereafter referring to any alcoholic beverage), using automatic or semi-automatic methods based on statistical processing (of the datamining type), optionally performed using artificial intelligence tools, as well as analytical data relating to the mineral composition, in particular the metal composition, of these beverages (wines).

To this end, this technology comprises a method, notably a computer-implemented method, for managing and/or monitoring:
the agricultural production of a raw material for preparing an alcoholic beverage, preferably wine;
the production of this alcoholic beverage;
the quality of the alcoholic beverage;
the organoleptic properties of the alcoholic beverage;
the authenticity of the alcoholic beverage; and/or
the traceability of the alcoholic beverage.

The invention also relates to a device for implementing this method.

### TECHNOLOGICAL BACKGROUND OF THE INVENTION

Alcoholic beverages, and in particular wines, have a significant economic impact. In 2020, the global market for alcoholic beverages corresponded to a turnover of 1.47 billion Dollars. Beers led the way, followed by spirits, wines and finally ciders. In the European Union, the combined annual sales of spirits and wines represented approximately 38 billion Euros in 2020.

In this field, consumer expectations relate to the quality of the alcoholic beverages, to health safety, to access to information relating to the production conditions and to guarantees in terms of the origin, identity and authenticity of these beverages.

The professionals in this sector are continuously seeking to improve the quality of the products, without neglecting any traditions. In this context, this involves limiting the risks of defects and proposing new sensations that are adapted as closely as possible to the tastes of consumers.

To this end, it is worthwhile having various and varied indicators, which allow the entire supply chain to be followed and monitored, from the production of the agricultural raw material, through to the production of the alcoholic beverage, until it is stored and aged in various containers.

Reference is made herein to the traceability, which is the ability to retrace the path of a food product throughout its production and distribution line, either from the original source of the product up until it is presented to the end consumer, or, as the saying goes, “from the farm to the table”.

In addition, the traceability is a requirement of the standards relating to the ISO 9000 & 9001 quality management system.

Traceability is also highly significant in terms of the health and safety of consumers. It allows them to be provided with reliable information relating to the substances present in the alcoholic beverages. It also guarantees that consumers are perfectly safe in terms of consumption, from the time of purchase to the end of life of the product.

Traceability is also crucial with respect to the origin, provenance and logistics of alcoholic beverages. It grants access to information relating to the vineyard site for producing the agricultural raw material of the alcoholic beverage, for example, wine, as well as relating to the production, storage and routing sites and conditions for the alcoholic beverage.

With respect to the identity and the authenticity of these alcoholic beverages, the 38 billion Euros figure for annual sales of wines and spirits in the European Union, in 2020, must be balanced against 1.3 billion Euros (that is, 3.3%) in losses due to the presence of counterfeit products on the market. These lost sales result in the direct loss of approximately 4,800 jobs in Europe.

Beyond the legal means for tackling this scourge, the stakeholders of this alcoholic beverages sector have implemented physical means for authenticating bottles, including RFID chips, indelible marking, electronic chips, holograms, theft-proof seals and barcodes.

The traceability for detecting counterfeits, falsifications or contaminations, also involves characterizing the alcoholic beverage by means of analytical chemistry, taken in its own right.

Alcoholic beverages, in particular wines, contain mineral elements, notably metals and metalloids. Wines usually contain (i) major elements such as Ca, K, Mg, and Na-10-1,000 mg/L-, (ii) minor elements: Al, Fe, Cu, Mn, Rb, Sr and Zn, −0.1-10 mg/L, and (iii) traces, among others, of Ba, Cd, Co, Cr, Li, Ni, Rb, and V −0.1-1,000 g/L.

There are multiple sources for the presence thereof: cultivation, vinification and growing. These elements influence the end quality and characteristics of the wine, at an oenological level: taste, flavors, evolution, quality, sensation, oxidation, etc., and can have a positive (or negative) impact on the sensations of consumers and the final selling price.

Long since overlooked, or passed on to simple regulatory positions relative to health standards to be observed (maximum concentrations to be observed), in favor of the multiple organic compounds of the wine, metals are nevertheless at the heart of the life of wine.

The use of the concentration of metals associated with predictive statistical analyses and/or other artificial intelligence tools has already been proposed in order to attempt to identify or trace families of wines.

However, these tools often offer inadequate performance capabilities, they have been developed based on low sample numbers (a maximum of a few hundred different wines), limited to a region and overlook many parameters. The results, even if they have made it possible to break from trends, often remain limited to specific regions and require highly specific analyses on the scale of ultra-traces and/or isotopic ratios of certain elements. Their performance capabilities are still insufficient and these approaches are still very rarely used, they often require very complex analyses in order to be reliable. They cannot be used for comparison, nor to establish wine categories or classifications, or to exclude a wine from well-defined wine categories.

### AIMS OF THE INVENTION

Within this context, the invention aims to meet at least one of the following aims:
providing an efficient method, notably a computer-implemented method, for managing and/or monitoring the production of agricultural raw materials useful for producing alcoholic beverages, the production of these alcoholic beverages, the quality of these alcoholic beverages, the organoleptic properties of these alcoholic beverages, the authenticity of these alcoholic beverages, and/or the traceability of these alcoholic beverages;
providing an efficient method, notably a computer-implemented method, for managing and/or monitoring wine-growing production, wine-growing production, the quality of the wines, the organoleptic properties of the wines, the authenticity of the wines, and/or the traceability of the wines;
providing an efficient and economical method, notably a computer-implemented method, for managing and/or monitoring the production of agricultural raw materials useful for producing alcoholic beverages, the production of these alcoholic beverages, the quality of these alcoholic beverages, the organoleptic properties of these alcoholic beverages, the authenticity of these alcoholic beverages, and/or the traceability of these alcoholic beverages;
providing an efficient and economical method, notably a computer-implemented method, for managing and/or monitoring wine-growing production, wine-growing production, the quality of the wines, the organoleptic properties of the wines, the authenticity of the wines, and/or the traceability of the wines;
providing an efficient, economical and reliable method, notably a computer-implemented method, for managing and/or monitoring the production of agricultural raw materials useful for producing alcoholic beverages, the production of these alcoholic beverages, the quality of these alcoholic beverages, the organoleptic properties of these alcoholic beverages, the authenticity of these alcoholic beverages, and/or the traceability of these alcoholic beverages;
providing an efficient, economical and reliable method, notably a computer-implemented method, for managing and/or monitoring wine-growing production, wine-growing production, the quality of the wines, the organoleptic properties of the wines, the authenticity of the wines, and/or the traceability of the wines;
providing an efficient, economical and reliable device for implementing the method targeted in the aforementioned aims.

### DESCRIPTION OF THE INVENTION

### Definitions

Throughout the present disclosure, any singular form equally denotes a singular or a plural form.

The definitions provided hereafter by way of examples can be used in order to understand the present disclosure:
“alcoholic beverage”: beverage prepared by alcoholic fermentation of a plant raw material, preferably an agricultural raw material. It particularly can be a wine, a beer, a cider, a liquor, a spirit, a whiskey, a brandy, a tequila, a vodka, a rum, a cognac, an armagnac, an Asian alcohol, etc.;
“inert container relative to the alcoholic beverage”: container that retains its physical integrity and good mechanical properties (tensile strength and plasticity still sufficient in order to be reliably used as a container containing liquid) after being in contact with the alcoholic beverage for 10 years and does not react with the alcoholic beverage, in particular polluting it with metallic elements;
“quality of the alcoholic beverage”: this notably covers the organoleptic properties, such as the taste, the smell, the structure, the texture, the balance, the color, the appearance, the consistency, amount of time in the mouth, etc., but also the health qualities independently of the toxicity associated with ethanol, such as the content of heavy metals such as lead or cadmium;
“substantially”, “of the order of”, “approximately”: means to + or − the nearest 10%, preferably 5%.

### Method

The invention meets at least one of the aforementioned aims and, according to a first aspect, relates to a method, notably a computer-implemented method, for managing and/or monitoring at least one factor
*f^{x}* selected from a set of factors comprising:
*f¹* the agricultural production of a raw material for producing an alcoholic beverage, preferably wine;
*f²* the production of this alcoholic beverage;
*f³* the storage of this alcoholic beverage;
*f⁴* the maturation of this alcoholic beverage;
*f⁵* the consumption of this alcoholic beverage;
*f⁶* the quality of this alcoholic beverage;
*f⁷* the authenticity of this alcoholic beverage relative to a reference selected from the group comprising, advantageously formed by: the names of the wine and the domains, the appellations of origin; the geographical indications; the traditional specialties guaranteed; the labels; the trademarks; and the combinations thereof;
*f⁸* the traceability of this alcoholic beverage;
*f⁹* the selling price of this alcoholic beverage;
said method mainly involving:
(a) collecting at least one, preferably at least two, samples of the alcoholic beverage, placing each of them in an inert container relative to the alcoholic beverage and sealably closing said container with an equally inert stopper;
(b) assigning data to each sample relating to the alcoholic beverage, which data is selected from the group comprising, advantageously formed by: data relating to the origin, data relating to the vineyard site, data relating to production, data relating to storage and maturation, data relating to consumption, physico-chemical data, qualitative data, in particular organoleptic data, economic data, commercial data, and combinations of these data;
(c) storing at least some of the samples collected in step (a) under determined conditions;
(d) analyzing each sample in order to determine at least one mineral profile, preferably a metallic profile;
(e) forming a database relating to the samples and derived from step (b) and step (d);
(f) optionally, completing and/or updating the data assigned in step (b), at least once, over all or some of the samples;
(g) optionally, completing and/or repeating the analyses performed in step (d), at least once, over all or some of the samples;
(h) processing these data by means of a statistical analysis, advantageously by means of automatic or semi-automatic methods based on statistical processing, and even more advantageously by “datamining”, preferably using artificial intelligence tools and/or other “datamining” techniques; and 20
using the processed data for managing and/or monitoring at least one of the aforementioned factors *f^{x}**.

The inventors are to be credited for having proposed, among other things, collecting, referencing, and storing samples of alcoholic beverages, for example, wine, and determining a reference mineral composition (for example, selected from among metals, metalloids, halogens, P, S, Se). Indeed, this principle according to the invention of storing samples of alcoholic beverages over a long period of time (several years) in order to have a reference is counter-intuitive. It is known in this field that alcoholic beverages are the focal point for chemical reactions and even often for biochemical reactions that give them an evolutionary character. This evolution can result in maturation in terms of an improvement in quality, but also to degradations that affect the organoleptic properties, or even the food safety of the considered alcoholic beverages. The inventors have nevertheless focused on the fact that the mineral composition of an alcoholic beverage benefits from stability over time, unlike the constituent organic compounds of this alcoholic beverage.

To this end, the inventors have wisely used inert, non-contaminating and barrier-forming containers, relative to the mineral profile of the alcoholic beverage, and moreover, containers each containing a sample of alcoholic beverages and able to be sealably closed with a stopper.

This allows the basic mineral composition of the samples and/or, at the very least, of the ratios of the basic mineral composition to be maintained over time.

This new and inventive approach grants access to a mineral profile, preferably a metallic profile, of the alcoholic beverage, for example, wine, that constitutes a reliable marker for controlling, managing and/or monitoring a plurality of steps of the entire supply chain, from agricultural production to commercial distribution, of the alcoholic beverage, as well as a certain number of states of the alcoholic beverage, before it is consumed. This reliable marker can be formed by several tens of different chemical elements, for example, more than 50 different chemical elements that provide a considerable wealth of information.

In the wine sector, the steps of the supply chain include the cultivation of the vine, the grape harvest and all the vinification processes, the growing, the maturation, the packaging (bottling) and the storage. The mineral (metallic) profile of the wines is also a reliable reflection of the health, regulatory, origin and authenticity characteristics, as well as of the quality and of the excellence of the wines.

The mineral profile (metallic) of the alcoholic beverage, of the wine, is of even more interest according to the invention, since it is refined/optimized by means of a statistical analysis, or even preferably by artificial intelligence algorithms, and since it is used as a tool for classifying and predicting the properties of the alcoholic beverage, in particular the vinous properties in the case of wine.

This subtlety in terms of characterization grants access to a whole range of improved statistical or predictive models for the alcoholic beverage.

With this in mind, for the producer of the alcoholic beverage this opens up a whole host of possibilities of corrective actions in terms of the agricultural production of the raw material, of the harvesting of this raw material, of the production of the alcoholic beverage from this raw material, of the storage and of the consumption of this alcoholic beverage.

In the field of wines, this results in notable oenological optimizations.
These new fields of action are extremely promising for this economic sector.

The method according to the invention also represents substantial progress in the pursuit of traceability, which makes it a formidable anti-counterfeiting weapon and an unrivalled means for monitoring the quality of alcoholic beverages, in particular wines.

It differs from existing methods in that it does not require any specific addition of products into the beverage and/or its containers and/or packaging and/or labelling.
Finally, it grants the producer of the alcoholic beverage better control of all the parameters of the supply chain, from agricultural production, the vineyard site, production, maturation, storage, to consumption. In other words, it provides the keys for reaching and attaining excellence.
The alcoholic beverages, in particular wines, that are obtained by implementing this method, undeniably have an increased commercial value.

Alcoholic beverages are characterized by a signature or a mineral profile, in particular a metallic profile. The origin of this profile, notably in the plant raw material, is its culture medium and/or its cultivation and wholly or partly the steps of producing the alcoholic beverage, namely, in the case of wine: vinification, growing, aging, maturation, storage, until it is consumed. This evolution of the mineral profile, in particular a metallic profile, results in the appearance and disappearance, via any variations in concentration, of the metallic elements of the alcoholic beverage.

In a first phase, the method according to the invention is based:
on the regular observation of this tracer, namely, the mineral profile (metallic), throughout its evolution;
on the prediction of this evolution;
as well as on the implementation of intervention means for controlling this evolution and bringing the alcoholic beverage to the desired quality.

In a second phase, the purpose of the method according to the invention is to use the profile of the metal signature of the alcoholic beverage for what it is, namely a tracer of the health and nutritional status and a marker of the identity of the alcoholic beverage.

### Step (a): Collecting

This step involves taking at least one, or even at least 2, samples, of at least 1 ml, for example, 50 ml, of the alcoholic beverage and placing each sample in an inert container relative to the alcoholic beverage, then sealably closing this container with a stopper, which is also inert relative to the alcoholic beverage.

Step (a): In a preferred embodiment of the method according to the invention, the number N of alcoholic beverages collected in step (a) is such that, in an ascending order of preference:

N≥500; N≥1,000; N≥10,000.

Preferably, the volume, Ve, of alcoholic beverage taken for each sample is such that, in an ascending order of preference, with Ve expressed in milliliters:

0.5≤Ve≤100; 1≤Ve≤50; 10≤Ve≤30.

Step (a): It is appropriate, according to the invention, for the inert container to be distinguished by its content, Tm, for each of the following mineral elements: Ca; Mg; Zn; Fe; Mn; Cu; Al; Si; Ni; V; Na; P; Co; Cr; K; Li; Pb; Se; Cd; Hg; As; such that, in an ascending order of preference, with Tm being measured according to a measurement method M^{tm} and expressed in ppb:

Tm≤5; Tm≤3; Tm≤2; Tm≤1.

The measurement method M^{tm} is described hereafter: the container is filled to more than 50%, and preferably between 60 and 75%, of its capacity, with 1% nitric acid, and is maintained at 50° C. for 12 hrs, the solution that is obtained is then sampled for an elementary analysis. The analysis is performed by means of an ICP-MS Agilent® 7700 appliance. This appliance is equipped with an octopole that will be used in helium collision mode (He flow rate=4.3 mL/min) before entering the quadrupole type mass analyzer. Each sample is then nebulized by means of a Micromist® nebulizer and then introduced into the ICP-MS appliance after passing through a Scott chamber, cooled to 2° C. A 60 second duration for balancing and stabilizing the signal is programmed before proceeding to the actual measurement.

Step (a): In accordance with a noteworthy feature of the invention, the container and its stopper are made from a material selected from among thermoplastic polymers, preferably polyolefins, and even more preferably from the group comprising, preferably formed by, polyethylene, polypropylene and mixtures thereof, with polyethylene being a preferred material for the stopper and polypropylene being a preferred material for the container. According to another possibility, the container could be made of silica.

According to an interesting modality of the invention, the samples are taken at different stages of evolution of the alcoholic beverage. For example, in the field of wines, samples are taken: while it is stored in a tank, before bottling and/or after bottling and/or at different times after bottling (several months or several years after bottling).

Advantageously, the container preserves the purity of the sample, in particular its inorganic purity and also effectively consolidates the data related to this sample. In order to guarantee the correct readability of this information over time, barcode/QR code labeling is an interesting solution. The data for the sample notably includes: the date, the sampler, the technician responsible for the analysis, the results, etc. They can be read, for example, by a simple “scan” in any conditions.

According to a noteworthy modality of step (a), taking a sample from a container, such as a tank, a barrel or a bottle, involves emptying at least 10%, preferably at least 1%, and at most 50%, of the amount of alcoholic beverage present in the sampling container, before taking the sample.

Step (b): The data assigned to the samples in this step are preferably:
the data relating to the origin that includes the name of the alcoholic beverage, the name of the producer, the name of the domain, the year of production, the sample collection date, the name of the cuvée, the batch number, and/or the type of alcoholic beverage;
the data relating to the vineyard site that includes the appellation of origin, the geographical indication, the country, the region, the site, the plot, the grape varieties, the distribution of the grape varieties, the exposure, the sunshine, the planting density (in feet/ha), the type of pruning of the vine, the cultivation mode, the fertilization of the vine, the green cover, the plant-health control, the watering, the average age of the vine, the relief, the type of soil, the source of the water, and/or the irrigation of the vine;
the data relating to production that includes the type of grape harvest, the date of the grape harvests, the type of sorting and destemming, the type of vinification, the type of press, the maceration time of the skins and seeds in the must, the material of the tanks, whether or not yeast is added, the type of bonding and clarification, the filtration system, and/or the blending;
the data relating to storage that includes the storage time, the successive container types, the container, the temperature, the humidity, the stopper type, and/or the packaging date;
the data relating to consumption that includes the presence and the content of sulfites, the presence and the content of phenolic compounds, the percentage of alcohol, and/or the presence and the content of aromatic compounds;
the qualitative data, notably organoleptic data, that includes evaluations of the Balance, the Length, the Intensity, the Complexity (and/or concentration) and the Type [BLIC (T) method];
the organoleptic data includes the color, flavors, tastes, and/or the duration of the impression of the flavors of the wine in the mouth, preferably expressed as cuadalies;
the economic data that includes the price, the sales volume, and/or the sales amount;
the commercial data that includes the labels, competition awards/medals, classifications, and/or received scores.
   These different types of data can be classified in files that will be organized in the database covered in step (e) of the method according to the invention.

It is advantageous for access to these sample-related data to be easy and fast over the entire lifetime of the sample.

### Step (c):

This step (c) involves gathering several samples of alcoholic beverages, in particular of different wines (more than 100, or even more than 1,000, or even more than 10,000) in a cabinet or storage room in order to establish a library of reference samples, which can be used for several years (for example, at least 5 years, or even at least 10 years). A sample library of alcoholic beverages, in particular of wines, is thus formed, in which library each sample retains its basic mineral content, notably its metal content. This stability opens the way to applications for health safety (heavy metal dosing, toxicity), for tackling fraud or counterfeiting or additional subsequent analyses, in association with the quality of a wine. Indeed, all the analyses are not necessarily performed shortly after taking samples, for economic reasons.

Advantageously, the storage conditions for the samples collected in step (a) are as follows: temperature≤40° C.; pressure≤1 at 2 bar; humidity≤90%; duration≥one month, preferably≥one year, and, even more preferably ≥5 years.

Step (d): It is preferable for this step to include the analysis of at least one reference metallic element, within a period that is less than or equal to 3 months, preferably that is less than or equal to 1 month, after sampling.

Step (d): The mineral profile analyzed in this step for the samples preferably comprises:
at least 5 mineral elements (called main elements) selected from B, Na, Mg, P, S, Cl, K, Ca;
at least the following metallic elements (called oligo-metals): Fe; Cu; Zn; Mn;
optionally at least one of the following metallic elements: Pb and Cd;
at least 10, preferably at least 30, and, even more preferably, at least 40 elements, for example, between 50 and 100, selected from the following trace mineral elements: Rb, Cs, Sr, Ba, Ce, Ti, V, Cr, Co, Ni, Zr, Mo, Ag, Al, Ga, Sn, As, Br, I, Se;
   and/or from the following ultra-trace mineral elements: La, Pr, Nd, Sm, Eu, Gd, Tb, Dy, Ho, Er, Tm, Yb, Lu, Th, U, Sc, Y, Nb, Ru, Rh, Pd, Hf, Ta, W, Re, Os, Ir, Pt, Au, Kg, TI, Bi, Sb;
optionally at least a portion of the isotopes of these elements;
the measurements of the concentrations of these elements; and/or
the ratios of concentrations of these elements, and, optionally, all or some of their isotopes.
   It should be noted that iron, copper, zinc and manganese are the main oligoelements that affect the life of the wine.
   According to one possibility of the invention, the analytical data can be consolidated on an analytical file specific to each alcoholic beverage (wine).

In order to optimize the method according to the invention, it is preferable that the elements selected for the analysis are the same for all the analyzed beverages.

Step (d): In this step, in addition to the absolute content of mineral elements, preferably of metallic elements, the relative contents between mineral chemical elements, preferably of metallic elements, are of interest. This relative analytical variant is notably suitable for countering dilution or evaporation effects. It also allows better sensitivity for detecting stable zones of metal concentrations of the wine, which zones would be less influenced by bottle contaminations and/or by precipitations that could lead to selective modifications of some metallic concentrations over time.

Step (d): In a preferred embodiment of the invention, where the alcoholic beverage is a wine, step (d) advantageously comprises analyzing chemical and physical parameters of the alcoholic beverage, with these parameters preferably being selected from the group comprising, advantageously formed by: ABV (Alcohol Strength by Volume), glucose+fructose, TA (Total Acidity), acetic acid, free SO₂, total SO₂, pH, active SO₂, ethanal, malic acid, lactic acid, CO₂, tartaric acid, gluconic acid, glycerol, optical density (absorbance at one or more wavelengths of 280, 420, 520 and 620 nm) and all the combinations of these parameters.

These chemical and physical parameters can undergo, in the same way as for the analytical data comprising at least one mineral profile of the alcoholic beverage, processing by means of statistical analyses according to step (h), optionally assisted by an artificial intelligence system.

It is particularly advantageous within the scope of the invention to check the relevance of the analyses of all or some of the relevant samples. To this end, comparative analyses can be performed of the concentration of at least one given target mineral element, between the undiluted sample and at least one dilution of this sample and/or between at least 2 dilutions of this sample. The solvent used for dilution advantageously is an aqueous solvent, for example, selected from the group comprising acidic aqueous solutions, such as nitric acid. The dilutions that are used can be: a 1v/Xv dilution and a 1v/2X dilution, with X being a positive natural integer ranging between 1 and 10. By way of an example, it is possible to have: a 1v/5v dilution and a 1v/10v dilution and/or a 1v/10v dilution and a 1v/20v dilution. According to the invention, the analysis of the concentration of the considered mineral element is considered to be reliable if the ratio between the C^{1v/Xv} concentration measured for the 1v/Xv dilution to the C^{1v/2Xv} concentration measured for the 1v/2Xv dilution, is such that: 1≤C^{1v/Xv}/C^{1v/2Xv}≤3; preferably 1.5≤C^{1v/Xv}/C^{1v/2Xv}≤2.5.

Step (e): Advantageously, the database formed during step (e) consolidates data relating to at least 500, preferably at least 1,000, and, even more preferably, to at least 10,000 different alcoholic beverages.

For each alcoholic beverage, for each wine, the database consolidates the analytical data of step (d) and the data assigned in step (b) relating to the origin, relating to the vineyard site, relating to production, relating to storage, relating to consumption, relating to quality, relating to organoleptic data, relating to economic aspects and relating to commercial aspects.
This database advantageously operates dynamically, by virtue of regular updates.

Step (h): Advantageously, processing the data according to step (h) mainly involves:
using at least one of the following means:
exploratory analyses and logistic regression for completing classifications based on a Principal Component Analysis (PCA);
discriminant analyses (or LDA (Linear Discriminant Analysis));
a predictive model algorithm, preferably selected from the group comprising, ideally formed by: Random Forest (RF) Decision Forests and/or Artificial Neural Networks (ANN) and/or Support Vector Machines (SVM);
performing at least one of the following actions:
identifying and predicting the origins of the various mineral elements within a wine;
identifying and predicting the impacts on the quality of a wine and the evolution of the quality of a wine of the various mineral elements within a wine;
adjusting the origins in order to adapt the quality, by selecting, for example, from the grape derived from plots including the best probability of a metallic profile corresponding to the desired quality.

### Discriminant Analyses:

The exploratory analyses and logistic regressions for completing classifications are based on Principal Component Analysis (PCA) or, depending on the field of application, the Karhunen-Loève Transform (KLT)1. This is a method that involves transforming variables that are linked to each other (referred to as “correlated” in statistics) into new variables that are decorrelated from one another. These new variables are called “principal” components, or principal axes. It allows the statistician to reduce the number of variables and to render the information less redundant.

This is both a geometrical (with the variables being represented in a new space, in directions of maximum inertia) and a statistical (with the search focusing on independent axes best explaining the variability, the variance, of the data) approach.

The Linear Discriminant Analysis (LDA) forms part of predictive discriminant analysis techniques. This involves explaining and predicting the membership of an individual in a predefined class (group) on the basis of their characteristics that are measured using predictive variables.

The linear discriminant analysis can be compared to the supervised methods developed in machine learning and to the logistic regression developed in statistics.

Such analyses can be performed by means of toolboxes or libraries in numerous software environments such as Python (Scikit Learn library), Matlab (Statistics and Machine Learning toolbox), R (statistical package for PCA, MASS package for LDA and package net for logistic regression).

The analysis techniques that are used are simple in this case, they rely on a database that is easy to understand, allowing the relationship to be understood between the selected elements since the techniques rely on a linear analysis and they are widely available in most statistical software packages.

### Predictive Model Algorithm:

The “Random Forest” (RF) decision forest analysis technique has been widely used in many scientific fields over recent years (Ga′al et al., 2012). This theory of statistical learning was proposed by Breiman in 2001 (Breiman, 2001; Tian et al., 2017). The random forests are formed by a group of predictor trees where each tree describes a subset of data, which have been sampled according to different observations and according to different variables (Breiman, 2001). The final prediction obtained by the classification forest is the majority vote obtained by consultation for all the decision trees (Tian et al., 2017; Zahiri et al., 2013). When the random forest operates in regression mode, the prediction is the average of all the predicted values (Palmer et al., 2007; Vigneau et al., 2018). These types of models can be used for highly varied applications, for example, for classifying invasive plant species (Cutler et al., 2007), for detecting fraud in Medicare (Bauder and Khoshgoftaar, 2018) or for predicting the progression of idiopathic pulmonary fibrosis using computed tomography (Shi et al, 2019).

### Artificial Neural Networks (ANN):

Artificial neural networks are machine learning methods that can improve their behavior with new observations, i.e., with experience (Anjos et al., 2015). ANNs are formed by an interconnected group of nodes (called artificial neurons) that process the information (Anjos et al., 2015). A neuron of the network operates according to a simple rule that combines its inputs at the output. For example, a neuron can compute the sum of its input signals and respond with an output signal by comparing whether this sum is greater than or equal to a threshold value. In the network, the organization of the connections allows different types of networks to be defined: proactive, recurrent networks, etc. Finally, the transmission of the signals from one neuron to the next can be modulated by learning rules (analogous to the neuronal plasticity of the central nervous system). In particular, the neurons can be connected in layers, namely, an input layer (which receives the input data), one or more intermediate layers and a final layer that generates the (variable) output signals (Anjos et al., 2015). It is also possible to work with “hidden” neurons (Linares-Rodriguez et al., 2013). They are capable of extracting significant features of the data and they can “learn” the relationship between the inputs and the outputs when sufficient training data is available (in terms of quantity and complexity) (Chiang and Chang, 2009). Here again, these models can be used for highly varied applications, for example, discriminating the botanical origin of various honey samples (Anjos et al., 2015), to modelling rain runoff (Chiang and Chang, 2009) in order to ultimately predict the best choice of tomato variety, their type of production and their date of harvest (Suarez et al., 2015), among others.

At present, current ANN models exist, which can be used in artificial intelligence using programming libraries, which are acknowledged as being efficient in many cases, and which do not require the in-depth development of a new optimal structure of the neural network. These methodologies therefore mean that it is possible to concentrate on analyzing data for the classification (discrimination) and for the prediction (regression).

### Support Vector Machines (SVM)

The SVM model uses the input data to construct a hyperplane (or hyperplanes) in a high-dimensional space, in order to perform classification, regression, or other tasks (RapidMiner, 2020a). In a classification problem, this involves determining the optimal separator hyperplane that maximizes the margin (distance between the hyperplane and the restricted subset of the samples closest to the hyperplane, which samples are also called “support vectors”. The SVM models can be applied in many applications such as diagnosing gear defects (Xing et al., 2017) or for assessing the state of roadways (Hadjidemetriou et al., 2018). The LibSVM library by Chang and Lin (Chang and Lin, 2011; RapidMiner, 2020a) was used to develop the SVM models for studying wines (Hsu et al., 2016).

Step (h): Preferably, the data processed in step (h) includes:
at least one mineral analytical profile, advantageously metallic, of the alcoholic beverage, measured in step (d) from the following elements:
at least 5 mineral elements (called main elements) selected from B, Na, Mg, P, S, Cl, K, Ca;
at least the following metallic elements (called oligo-metals): Fe; Cu; Zn; Mn;
optionally at least one of the following metallic elements: Pb and Cd;
at least 10, preferably at least 30 and, even more preferably, at least 40 elements selected from the following trace mineral elements: Rb, Cs, Sr, Ba, Ce, Ti, V, Cr, Co, Ni, Zr, Mo, Ag, Al, Ga, Sn, As, Br, I, Se;
and/or from the following ultra-trace mineral elements: La, Pr, Nd, Sm, Eu, Gd, Tb, Dy, Ho, Er, Tm, Yb, Lu, Th, U, Sc, Y, Nb, Ru, Rh, Pd, Hf, Ta, W, Re, Os, Ir, Pt, Au, Kg, TI, Bi, Sb;
optionally at least a portion of the isotopes of these elements;
the measurements of the concentrations of these elements; and/or
the ratios of concentrations of these elements, and, optionally of all or some of their isotopes;
and at least 1, preferably at least 5, physicochemical parameters of the alcoholic beverage selected from the group of parameters comprising, advantageously formed by: ABV (Alcohol Strength by Volume), glucose+fructose, TA (Total Acidity), acetic acid, free SO₂, total SO₂, pH, active SO₂, ethanal, malic acid, lactic acid, CO₂, tartaric acid, gluconic acid, glycerol, optical density, and all the combinations of these parameters (preferably measured less than one week before or after bottling, or at a determined time in relation to bottling).

Step (i): According to a first possibility, the use of the data processed in step (h) for managing and/or monitoring the agricultural production (factor *f¹*) of the raw material for producing the alcoholic beverage mainly involves:
identifying at least one parameter, Pa^{p=1 to z (z: natural integer)}, or a linear combination of these parameters, allowing the quality of the alcoholic beverage to be improved;
identifying, in the cultivation and/or harvesting processes, one or more modalities for influencing at least one parameter Pa^{p=1 to z (z: natural integer)};
modifying the one (or more) identified modality(ies) in order to change the parameter Pa^{P=1 to z (z: natural integer)} in terms of an improvement in the quality of the alcoholic beverage.

Step (i): According to a second possibility, the use of the data processed in step (h) for managing and/or monitoring the production of the alcoholic beverage (factor *f²*) mainly involves:
identifying at least one parameter, Pa^{p=1 to z (z: natural integer)}, or a linear combination of these parameters, allowing the quality of the alcoholic beverage to be improved;
identifying, in the vinification and/or blending and/or growing processes, one or more modalities for influencing at least one parameter Pa^{p=1 to z (z: natural integer)};
modifying the one (or more) identified modality (ies) in order to change the parameter Pa^{p=1 to z (z: natural integer)} in terms of an improvement in the quality of the alcoholic beverage.

Step (i): According to a third possibility, the use of the data processed in step (h) for managing and/or monitoring the storage (factor *f³*) and/or the maturation (factor *f⁴*) and/or the consumption (factor *f⁵*) of this alcoholic beverage mainly involves:
identifying at least one parameter, Pa^{p=t to z (z: natural integer)}, or a linear combination of these parameters, allowing the quality of the alcoholic beverage to be improved;
identifying, in the storage and/or maturation and/or consumption processes, one or more modalities for influencing at least one parameter Pa^{p=1 to z (z: natural integer)};
modifying the one (or more) identified modality (ies) in order to change the parameter Pa^{p=1 to 2 (z: natural integer)} in terms of an improvement in the quality of the alcoholic beverage.

Advantageously, the parameter Pa^{p=1 to z (z: natural integer)} is selected from the group comprising, ideally formed by: the tasting parameters, preferably, balance, length, intensity, complexity, concentration and/or type; the overall composition parameters, preferably, alcohol strength by volume, glucose and fructose, total acidity, acetic acid, free SO₂, total SO₂, pH, active SO₂, ethanal, malic acid, lactic acid, CO₂, tartaric acid, gluconic acid and/or glycerol; the parameters of contents of specific aromatic and/or coloring molecules.

Step (i): According to a fourth possibility, the use of the data processed in step (h) for managing and/or monitoring the quality (factor *f⁶*) of the alcoholic beverage mainly involves implementing the method according to the invention in accordance with at least one of the 3 possibilities described in the preceding paragraphs.

Step (i): Within the context of this fourth possibility, it is possible to contemplate, according to a noteworthy variant of the invention, the use of the data processed in step (h) for managing and/or monitoring the quality (factor *f⁶*) of the alcoholic beverage, mainly involving:
identifying one or more mineral profiles, preferably metallic profiles, each forming a specific target signature of a certain level of quality for an alcoholic beverage or an alcoholic beverage promised to be at a certain level of quality;
searching for and selecting from a group of alcoholic beverages, the one or more alcoholic beverages for which the mineral profile, preferably a metallic profile, corresponds to a target signature;
marking this or these selected beverages using an acquired or forthcoming quality assurance label;
optionally using the mineral profiles, preferably metallic profiles, of the non-selected alcoholic beverages, to anticipate negative evolutions of these beverages and to provide the necessary corrective solutions.

The method according to the invention thus allows reliable, and optionally advance, detection, from among alcoholic beverages, for example, wines, during growing (tank to barrel) and/or during maturation/storage (barrel, cask or bottle), of actual nectars or of future nectars.

The invention thus offers a screening means for selecting alcoholic beverages, for example, current or forthcoming high quality wines.

It is thus possible to grant the selected alcoholic beverages, for example, wines, current or forthcoming quality guarantee labels, which will result in their commercial value being mechanically increased.

Step (i): According to a fifth possibility, the use of the data processed in step (h) for managing and/or monitoring the authenticity (factor *f⁷*) of the alcoholic beverage mainly involves:
identifying one or more metallic profiles forming specific signatures of the origin of the alcoholic beverage;
using this or these signatures as markers guaranteeing the authenticity of the alcoholic beverage;
detecting counterfeits using these markers.

Step (i): According to a sixth possibility, the use of the data processed in step (h) for managing and/or monitoring the traceability (factor *f⁸*) of the alcoholic beverage mainly involves:
identifying one or more metallic profiles forming specific signatures of the path of the alcoholic beverage, from its source to its consumption, via its production, its packaging and its storage;
using this or these signatures as tracers of the alcoholic beverage, notably within the context of surveys relating to food or health quality incidents.

The method according to the invention thus grants access to traceability for alcoholic beverages, in particular wines, for the greatest benefit of the health and safety of consumers, from the time of purchase to the end of the lifetime of the beverage.

By virtue of this traceability, the consumer and the producer have plenty of information available concerning the substances present in the beverage, as well as concerning the origin and the conditions for producing and conveying this beverage.

Step (i): According to a seventh possibility, the use of the data processed in step (h) for managing and/or monitoring the selling price (factor *f⁹*) of the alcoholic beverage mainly involves:
identifying one or more metallic profiles forming (one of) the specific signatures of an average selling price of the alcoholic beverage in its category;
using this or these signatures as indicators of accessible ranges of selling prices and anticipating the future selling prices of the alcoholic beverage.

Step (i): Pa^{p=1 to z (z: natural integer)}

Pa^{p=1 to z (z: natural integer)} is a parameter linked to the execution modalities of the supply chain of the alcoholic beverage (for example, wine), by virtue of which the data processed in step (h) allows corrective actions to be set up for these modalities, in order to improve the quality of the alcoholic beverage (for example, wine).

In the case of wine, and according to a preferred embodiment of the invention, this parameter Pa^{p=1 to z (z: natural integer)} can relate to tasting Pa¹, the overall composition Pa³ and the content of aromatic molecules Pa³ and/or specific colorings Pa⁴.

### Pa¹: Tasting (Balance, Length, Intensity, Complexity and Type)

The BLIC (T) method meaning: Balance-Length-Intensity-Complexity (and/or Concentration)-Type. Each parameter is added to the others in order to estimate the general quality of the wine.

The Balance is fundamental. It is the main component of quality. The term “balance of a wine” is generally understood to mean a harmony between the various components of the texture in the mouth: acidity, alcohol and lubricity, tannins and sugars, etc. It is often easier to define an unbalanced wine. It is a wine in which one of the components is excessively or insufficiently present in the wine. Thus, a wine that is too green with sharp and acerbic acidity, or an over extracted wine with astringent and bitter tannins, will be unbalanced wines. An unbalanced wine is generally deemed to be unsatisfactory (or mediocre) by tasters.

The Length of the wine is understood from the aromatic point of view, and it is preferable for the flavors of the wine to remain in the mouth for a long time once it has been swallowed (or spat out in professional tasting). A long-lasting wine in the mouth is often referred to when the flavors of a wine can be perceived for many seconds, or minutes.

The Intensity is also linked to the flavors of the wine. The more scented the wine, the more intense it is said to be. Fine wines are thus often highly aromatic, although this is not always sufficient for making high-quality wines (for example, a cheap wine by Gewurztraminer from the Alsace can be aromatically intense, but have a relatively short length, for example).

The Complexity is also a quality parameter. It expresses the number of flavors that can be detected by smelling the wine. The richer the wine in terms of different flavors, the higher quality it is considered to have. A simple entry level muscadet from the Loire is always less complex than a Bourgogne Meursault. For some wines, maybe even young wines, which have not yet revealed their entire aromatic potential, it is then possible to focus on the concentration of the flavors. The richer a wine, the higher quality it is deemed to have.

Finally, the Type (or identity) (or Vineyard site) is the most complex concept to be judged. The type of a wine can be understood to be a unique and recognizable character of its site of production. Of course, it is by tasting wines from all the regions of the world that the vineyard sites are gradually recorded into memory. A typical wine is a wine that unmistakably has a “taste of the site”. When assessing the quality of a wine, the type is often of assistance for fine and subtle wines, which may not be as expressive and aromatic as certain “bombshells” from the new world, developed from aromatic grape varieties. A Muscadet Sur Lie, due to its vineyard site, is, for example, never highly concentrated. However, it has a crystalline freshness, with this saline minerality contributing to its typical and recognizable taste, in short, it is highly distinctive.

Once the richness of each of the BLIC (T) parameters has been assessed, it is possible to better identify the quality of the wine. An unbalanced wine is a mediocre, or even defective, wine (if the smells are not clear or are unpleasant). A simply balanced wine is considered to be acceptable. With an additional feature (for example, significant concentration), it is considered to be good; with two additional features, it is considered to be very good. Furthermore, if all the parameters are present, the tasters can then assess its quality as being excellent. An excellent wine is therefore equally balanced, aromatically intense, with complex flavors, with a good length or even a certain identity.

### Pa²: Overall Composition

This involves analytical data for determining the overall composition of a wine and remaining within the appropriate field for a good level of quality. These analytical data are selected from the group comprising, advantageously formed by: ABV (Alcohol Strength by Volume), glucose+fructose, TA (Total Acidity), acetic acid, free SO₂, total SO₂, pH, active SO₂, ethanal, malic acid, lactic acid, CO₂, tartaric acid, gluconic acid, glycerol, and all the combinations of these data.

These analytical data are acquired using the chemical analysis methods notably provided by the current standards in the field of wines, such as, for example: FTIR (Fourier Transform InfraRed spectroscopy), automated visible spectroscopy, colorimetry, capillary electrophoresis, titrimetry, potentiometry, etc.

### Pa³: Aromatic Molecules

Some of these aromatic molecules have a positive effect in terms of taste, others have a negative effect or are considered to be defects and/or contaminants, and others significantly contribute to the nose of the wines.

It is therefore possible to contemplate, in accordance with the invention, setting up, with respect to the data processed in step (h), by means of a statistical analysis and, optionally, with the assistance of artificial intelligence, corrective actions aimed at:
(i) increasing, and/or adjusting and/or stabilizing, the amount of aromatic molecules having a positive taste effect, over time;
(ii) decreasing the amount and breaking down and/or neutralizing, the aromatic molecules with a negative taste effect or considered to be defects and/or contaminants, over time;
(iii) adjusting the amount of aromatic molecules involved in the nose of the wines.

The following can be cited as examples:
aromatic molecules of the following type (i): esters (acetaldehyde (fresh apple), isoamyl acetate (banana), ethyl acetate (acescent character), ethyl butyrate, ethyl isobutyrate, ethyl 2-hydroxy-4-mepentanoate ethyl butyrate, ethyl octanoate, ethyl decanoate, ethyl hexanoate, ethyl isovalerate, isoamyl acetate, 2-phenylethtnanol, 3-isobutyl-2-methoxypyrazine (IBMP) and isopropyl-methoxypyrazine (IPMP), sec-butyl-methoxypyrazine (SBMP), green pepper and plant flavors, terpenes and norisoprenoids (terpenols: geraniol, linalol, α-terpineol, nerol, citronellol, beta-damascenone, alpha-ionone, betaionone), wood flavors (trans-whiskylactone, cis-whiskylactone (coconut, fresh wood), eugenol, isoeugenol (clove), acetovanillone, vanillin, ethyl-vanillin, ethyl-vallinate (vanilla), furfural, methyl furfural (toasted bread, toasted almond), methyl-guaiacol, gaiacol (toasted bread, smoked), syringol, 4-methyl-syringol, 4-allyl-ssyringol, acetosyringone (smoked), syringaldehyde (smoked, hints of vanilla), o-cresol (smoked, burnt), trans-nonenal (green wood), maltol (caramel, cotton candy);
aromatic molecules of the following type (ii):
Molecules with high odorisity, predominantly responsible for the tastes of the stopper or of mustiness present in the wines: volatile phenols (ethyl-4-phenol (stable, leather), ethyl-4-guaiacol (spicy), vinyl-4-phenol (gouache, burnt rubber), vinyl-4-guaiacol (clove), haloanisoles [trichloroanisole (TCA), tetrachloroanisole (TeCA) and pentachloroanisole (PCA)], halophenols [trichlorophenol (TCP), tetrachlorophenol (TeCP), pentachlorophenol (PCP) and tribromophenol (TBP)];

Geosmine (high-odoristy compound, which has a very marked earthy-mustiness smell):
3 molecules responsible for mousiness: (popcorn, cooked rice, mouse urine, pet store, etc.), 2-acetyl-tetrahydropyridine (ATHP), 2-Acetyl-1-pyrroline (APY), 2-ethyl-tetrahydropyridine (ETHP);
mycotoxins [ochratoxin A (OTA)] and biogenic amines [Histamine, Methylamine, Ethylamine, Tyramine, Phenylethylamine, Putrescine, Isoamylamine, Cadaverine];
benzaldehyde (benzoic aldehyde) with a bitter almond smell, and benzyl alcohol (benzyl alcohol derived from the plasticizer present in the epoxy resin coatings forming some packaging for bottling, enters the wine, where it is oxidized as benzaldehyde);
2-bromo-4-methylphenol (iodine tastes (sometimes of oyster)), ethyl carbamate, diethylene, monopropylene and monoethylene glycol;
molecules responsible for the smoky taste (cold ash flavors and an acrid tannin character), free forms: o-cresol, gaiacol, 4 methyl-gaiacol, syringol, 4 methyl-syringol, 4 allyl-syringol or the glycosylated precursors: glycosylated gaiacol, glycosylated gaiacol, glycosylated 4-methyl-gaiacol, gaiacol-glucopyranoside, gaiacol-gentiobioside, gaiacol-rutinoside, 4-methyl-gaiacol-rutinoside;
type (iii) aromatic molecules: propan-1-ol, 2 methylpropan-1-ol, isopentanols, 2-methyl-butanol, 3-methyl-butanol, butan-1-ol, butan-2-ol, but-2-ene-1-ol.

### Pa³: Coloring Molecules

It is possible to contemplate, in accordance with the invention, setting up, with respect to the data processed in step (h), by means of a statistical analysis and, optionally, with the assistance of artificial intelligence, corrective actions aimed at adjusting the quantity and/or the evolution of the quantity over time, of coloring molecules acting on the appearance of the color.

The following can be cited by way of examples: tannins and anthocyanins, resveratrol. Resveratrol is a polyphenol predominantly present in the skin of grapes. The richness of resveratrol depends on the grape variety (Pinot Noir, Grenache, Mourvèdre and Merlot contain more), the vinification, the geographical origin and exposure to cryptogamic diseases. This powerful antioxidant can have beneficial effects on human health.

### Step (i):

Modalities of the Cultivation and Harvesting Process for Influencing at Least One Parameter Pa^{p=1 to z (z: natural integer)}-, Preferably Pa¹, Pa² and/or Pa³.

In particular, this can involve selecting the location of the vines, the type of vines, the type of cultivation, the type of grape harvest and finally the date of the grape harvest and the state of maturity of the grapes at the time of the grape harvest.

The location, for example, with the GPS coordinates, of the vine plots can be widened to the fields, appellation zones, community, regions. The selected location also determines the relief [Flat-Slope-Steep Slope (>20°)—Extreme Slope (>30°)], the exposure/sunshine and the type of soil: acidic, basic, clay-calcareous-gravel/sand-marl-chalk-granite-roundstone-shale-siliceous-laom-sand-gneis-sandstone-others.

The type of vine is defined by the grape varieties (type and estimated distribution as a %), the age of the layouts, the planting density.

The cultivation can be integrated, conventional, biological, or biodynamic. It also can be notably defined by the following elements:
planting density (in feet/ha);
fertilization of the vine (none-binary fertilizer KMg-organic fertilizer-mineral fertilizer);
green cover (non-green cover-green cover every other row-green cover on all the inter-rows);
other (spontaneous winter cover-seeded winter cover-spontaneous permanent cover-seeded permanent cover);
plant-health control (none-semi-dose-diseases-pests-herbicides) in particular the amount of copper spread/Ha;
type of pruning of the vine [gobelet-royat cordon-single guyot-double guyot-lyre-precision mechanical pruning (PMP)];
irrigation of the vine (furrow-foliage spraying-ground spraying-localized (droplets)):
water source (if irrigation is present): rain-reserves-groundwater-water course;
average age of the vine: number of years-old vines-young vines;
vintage: (and associated climate in the production zone).

Harvesting or grape harvesting offers numerous adjustment variables, including:
date of grape harvest: associated climate 1 week before and during;
state of maturity of the grapes at the time of the grape harvest and in particular the maturity checking parameters that are often analyzed: density, glucose+fructose, probable degree, acquired ABV, AT, volatile acidity, pH, malic acids, tartaric, gluconic, citric, glycerol: state of health indicators, potassium, ammoniacal nitrogen, nitrogen α-amino nitrogen, total assimilable nitrogen, index delta C-13: water stress indicator.
Type of grape harvest: manual-mechanical.

### Step (i):

Modalities of the Vinification and/or Blending and/or Growing Process for Influencing at Least One Parameter Pa^{p=1 to z (z: natural integer)}-, Preferably Pa¹, Pa² and/or Pa³.

These modalities relate to several stages of the production of the alcoholic beverage, that is wine, corresponding to a particular embodiment of the method according to the invention, namely: receiving the grape harvest and pre-fermentative operations, alcoholic fermentation and fermentative operation, vatting and alcoholic fermentation, growing and operation following vinification, end-of-vinification decision-making and checking alcoholic and malolactic fermentation monitoring.

Receiving the grape harvest and prefermentative operations
Sorting upon receipt of the grape harvest: manual-mechanical;
Destemming: type of destemmer;
Grape crusher: pre-fermentative grape crusher completed (to release the juice more easily);
Pressing (pressing berries for white wine vinification), types of press (mechanical-hydraulic-pneumatic-horizontal-vertical);
Marc pump (large endless screw), type and materials of the screw;
Wine tank material used (stainless steel-concrete-wood-ceramic);
Volume and tank filling rate (important for oxygenation);
Maceration time of the skins and seeds in the must: for example, as a number of days;
Adding after filling the tank, oenological corrections: sulfiting, chaptalization, concentration by reverse osmosis, acidification, deacidification, dealcoholization, enzyming, wood chips;
Extraction and/or addition of juice (juice can be removed in order to increase the skin ratio of the grape/juice, in order to obtain more concentrated wines, any loss is then used for rosé or a less qualitative batch);
Management of the clarification and of the aromatic properties: possible stabulation (for white and rosé vinification) increases the expression of the flavors;
Settling (clarifying the must by removing the suspended particles and the various impurities, notably for white and rosé wines);
Possible blending of different musts (in the case of the vinification of different grape harvests, plots, etc.);
Skin maceration (for white and rose vinifications for direct pressing;—contacting the skins and the must for a few hours in order to cause the primary flavors and the anthocyanins to diffuse (for rose wines).

### Alcoholic Fermentation—Fermentative Operations

Operations prior to vatting: carbonic maceration (essentially for primeur wine) from whole grapes (not destemmed and not crushed) allowing alcoholic fermentation to start in the grape berry;
Pre-fermentative cold maceration (5-15° C.) with whole grapes allows alcoholic fermentation to start in the grape berries);
Crushing (crushing can occur before the vinification itself or even afterward);
Optionally, skin maceration [this operation also allows natural yeasting from the indigenous yeasts present on the skins of the grape berries (which enhances the identity of the wine)];
Carbon maceration or Pre-fermentative cold maceration.

### Vatting—Alcoholic Fermentation

Pure indigenous yeasts or the addition of yeast (selected by laboratories with high fermentative potential);
Temperature and thermoregulation time for the tanks;
Daily reading of the density of the must (more dense sugar than alcohol) and possible additions;
Possible techniques for improving and extracting flavors, color and tannins: trapping (between 8 and 20 days depending on the wines) involving forcing the marc cap into the liquid part of the must during fermentation while emitting it in order to promote the diffusion of phenolic compounds and flavors);
Reblending (recovery of the must undergoing fermentation accumulated in the bottom of the tank in order to transfer it onto the marc cap that floats on the surface of the tank);
Offloading involving recovering all the must undergoing fermentation accumulated in the bottom of the tank and transferring it into a second tank. It is then returned to the marc cap, which has compacted and is drained at the bottom of the tank in order to improve the maceration), optional hot maceration of the marc tank by transiently dissociating it (1 to 2 days) from the rest of the tank;
Optionally maceration under hot conditions, mutage (for specific wines stopping the fermentation in order to obtain a natural soft wine, possible clacking (or macro-oxygenation) for briefly and occasionally adding an amount of wine), aromatization, etc.
Upon completion of fermentation, the juice is made up of a large amount of alcohol, which accentuates the extraction and notably that of undesirable compounds, the extraction will then be kept to a minimum, it will be left to simmer. The marc cap is now only minimally watered (very limited reblending) on a daily basis, or even, every two days, in order to renew the juice present in the marc and to prevent it from turning sour. At this time, tasting is of utmost importance, when the cellar master and the oenologist consider that the material and the fat have been sufficiently extracted, the tank is drained in order to draw out any wine that is bitter, green and/or dry. The vatting time varies as a function of the quality of the grapes and of the desired wine, it generally oscillates from 10 to 30 days.

### Operations Post-Vinification—Growing

Malolactic fermentation generally occurs after alcoholic fermentation. It allows the acidity of some wines to be reduced.
Sulfiting wine allows it to be protected against oxidation and subsequent potential microbiological deviations, it occurs after the malolactic fermentation so as not to impede it.
The wine is then grown with possible micro-oxygenation and/or macro-oxygenation.
Growing can be implemented in various ways:
growing in a tank—growing wines in wood—type of tank—times and conditions; growing the wine with micro-oxygenation and/or macro-oxygenation: oxygenation conditions.
Optionally, the following operations are finally implemented before bottling:
   clarification and stabilization;
   tartaric stabilization;
   filtration [conventional filter with cellulose or earth plates-Tangential Micro-Filtration System (TMF)]; and/or
   bonding [bentonites-fish glue-casein-egg albumin-gelatin-PVPP-soils and silica gels, etc.].

Some of the correction operations of the method according to the invention notably involve acting on:
bonding of the white and rose wines (protein stabilization bonding, stabilization and clarification bonding, ferrocyanide treatments);
the stability of the white and rose wines (Pinkink for white wines, protein stability, tartaric stabilization, treatments for tartaric stability); and/or
bonding of the red wines (stabilization and clarification bonding, tartaric stability, clogging index and behavior during filtration).
   Upon completion of vatting, the flow of the first-press wine under the effect of gravity, the drawing off and then the pressing of the marc, yield the press wine.
   Blending can be performed at any time, between the first-press wine and the press wine, between two cuvées, between varietal wines in order to produce a blended wine.

### End-of-Vinification Decisions (Analyses)

Controlling and monitoring alcoholic and malolactic fermentation is based on the following indicators: glucose+fructose, acquired ABV, probable degree, AT, acetic acid, free SO₂, total SO₂, pH, malic acid, lactic acid.

A report is completed after drawing off, based on the following indicators: total SO₂, pH, active SO₂, ethanal, malic acid, lactic acid, CO₂, tartaric acid, gluconic acid, glycerol, iron, copper (whites and rosés)—tasting OTA index, coloring intensity, on demand).

During bottling or BIB (Bag-In-Box), the following are dosed: ABV, glucose+fructose, AT, acetic acid, free SO₂, total SO₂, pH, active SO₂, ethanal, malic acid, lactic acid, CO₂, tartaric acid, iron, copper, 2 tests of protein stability (white-rosé), tasting, a microbiological analysis is performed using cytometry, and, optionally, a cold test and/or a clogging index is performed on demand.

Step (i): According to a noteworthy modality of the invention, step (i) of the method can include a sub-step (ic) involving a corrective action of the mineral profile that can involve:
extracting metals before bottling using filtration, preferably tangential filtration associated with polymers complexing the metals (placed on the other side of the membrane relative to the wine); and/or
extracting metals in a step before bottling or after opening the bottle, by trapping, preferably using a biopolymer gel functionalized by ultra-chelating agents placed directly in contact with the wine.

The method according to the invention takes advantage of focusing on the mineral/metallic analysis of alcoholic beverages, in particular wines, using suitable containers for collecting samples, by processing the data statistically, preferably by targeting learning processes based on artificial intelligence and by collecting the data gathered in a database provided to this end.

This notably allows the impact of metals on the tastes of alcoholic beverages (for example, wines) to be known, and does so by introducing analytical objectivity into all the scores, classifications and oenological competitions or other professional alcoholic beverage competitions.
The alcoholic beverages, in particular wines, thus can be mutually discriminated, as a function of their quality at an instant t, but also by taking into account the perspectives in terms of the evolution of this quality, in view of the mineral profile (for example, metallic) of the alcoholic beverage. The fact that there is access, by virtue of the method according to the invention, to the evolutionary potential of the alcoholic beverage is a new criterion that is highly advantageous, which facilitates its evaluation and allows scores to be assigned with greater accuracy and impartiality. The rapid analysis of the data from the database by virtue of the methods and the tools according to the invention provides effective assistance for making an informed decision, and especially an objective decision, for classifying or evaluating alcoholic beverages, notably wines, but also upstream in order to provide all the preventive and/or corrective actions that are desirable at every stage of the preparation of alcoholic beverages.

### Device

According to a second aspect of the invention, the invention relates to a device for implementing the method according to the invention, characterized in that it comprises a sample library comprising at least one enclosure that houses and stores the samples collected in step (a) in inert containers each closed by a stopper, and in that this enclosure is able to place these samples under given temperature, pressure, humidity, and atmospheric conditions.

A further aim of the invention is a system for the computerized management of the sample library and of the database.

### DESCRIPTION OF THE FIGURES

The appended figures illustrate non-limiting embodiments, in which:

FIG. 1 shows a distribution of the wines analyzed in the examples, according to their grape varieties;

FIG. 2 shows a distribution of the wines analyzed in the examples, according to their origins, their regions;

FIG. 3 shows a distribution of the wines analyzed in the examples, according to their cultivation modes;

FIG. 4 shows a graph, in which:

the ordinate corresponds to the number of wines analyzed in the examples that fall within a price category and that are counted; and
the abscissa corresponds to the concentration of Ci expressed in μg/L;

FIG. 5 shows a graph, in which:
the ordinate corresponds to the number of wines analyzed in the examples that fall within a category (red wine or white wine) and that are counted; and
the abscissa corresponds to the concentration of K expressed in μg/L;

FIG. 6 shows a graph, in which:
the ordinate corresponds to the number of wines analyzed in the examples that fall within a price category and that are counted; and
the abscissa corresponds to the concentration of K expressed in μg/L;

FIG. 7 shows a graph depicting the distribution of the concentrations of Mg of wines analyzed in the examples, as a function of the quality of the wine (in this case expressed by a selection of the obtained scores);

FIG. 8 shows a graph depicting the distribution of the concentrations of K of wines analyzed in the examples, as a function of the quality of the wine (in this case expressed by a selection of the obtained scores);

FIG. 9 shows a graph depicting the distribution of the concentrations of Ca of wines analyzed in the examples, as a function of the quality of the wine (in this case expressed by a selection of the obtained scores);

FIG. 10 shows a graph depicting the distribution of the concentrations of Na of wines analyzed in the examples, as a function of the quality of the wine (in this case expressed by a selection of the obtained scores);

FIG. 11 shows a matrix of the statistical correlation between the analyzed mineral elements (lanthanides and W, S, Nb);

FIG. 12 shows the mineral profile of a wine of the 2^{nd} series of examples, according to a star-shaped graphical representation (Kiviat diagram);

FIG. 13 shows a graph providing the distribution of the metallic and mineral elements over the various classes of concentrations of the wines of the 2^{nd} series of examples;

FIGS. 14 to 17 show the mineral profiles depicted as Kiviat diagrams for red, white, rosé, and sparkling wines of the 3^{rd} series of examples.

### EXAMPLES

### 1st Series of Examples: Paragraphs to [0140] to [0220]

Sampling [step (a)] and storage [step (c)] of samples.

The samples are taken directly from 24 commercial wine bottles:
1—Saint Joseph-Les Caves Saint Ronain—Jacques Delorme
2—Macon Villages-Domaine du grison
3—Hermitage—E. Guigal
4—Pays d′Oc—Naturae Chardonnay—Gerard Bertrand
5—Veneto—Cantine Maschio—Verduzzo
6—Montagne Saint Emilion—Chateau Petit Clos du Roy-François Janoueix
7—Montagne Saint Emilion-Chateau Petit Clos du Roy (Premium)-François Janoueix
8—Pomerol—Château I'Évêché-François Janoueix
9—Saint Emilion grand cru—Château Condat—François Janoueix
10—Meursault—Domaine Saint Marc—Sous la Velle
11—Bourgogne Aligoté—Nuiton Beaunoy
12—Bourgogne—Nuiton Beaunoy—Pinot Noir
13—Mâcon—Le domaine du Grison—Mâcon Péronne
14—Côtes du Rhône—E,Guigal
15—Chassagne Montrachet—Domaine Paul Pillot—1er Cru Les Champs Gains
16—Côte du Rhône—Cellier des Dauphins—Vieilles Vignes
17—Montagny—Domaine de la Guiche—Montagny 1er Cru
18—Chassagne Montrachet—Jean Bouchard
19—Côte-Rôtie—Domaine Vernay—Gisèle
20—Bourgogne Passe tout grains—Bernard Millot—100 Noms
21—Nuits Saint Georges—Domaine Henri GOUGES—Villages Rouge
22—Maranges—Michel Sarrazin et Fils—Côte de Beaune
23—Givry (Bourgogne rouge)—Michel Sarrazin et Fils—Givry 1er cru—Les vieilles vignes
24—Gevrey Chambertin (Bourgogne rouge)—Domaine Denis MORTET—Mes Cinq Terroirs

Once the bottle is open, a portion of the wine is emptied out (at least 100 ml and less than half the contents of the bottle), then two “metal free” storage vials (ultra-low tube MetalFree™ Centrifuge Tubes 15 ml, ref. 3134-345-Labcon®) are filled with the wine (at least 10 ml per vial). This step is performed less than one hour after opening the bottle. Previously emptying a portion of the bottle avoids any surface deposits and washes any dust from the flow area that could be in contact. Still leaving at least half the bottle before sampling minimizes sampling any deposits at the bottom of the bottle.

The sample tubes are then placed in a storage zone at ambient temperature.

Some of the contents of one of these sample tubes is sampled in order to perform ICP-MS metallic content analyses (see step (d) below, paragraphs [0143]-[0149]).

[Step (b)] Each tube is referenced and the information available concerning the wine is entered into a data table. This information is listed below in the passage relating to step (e) for generating the database.

[Step (d)] Analysis of the content of mineral elements and metallic traces

The reagents that are used are of ultra-pure quality: HNO₃ 35% (Suprapur, Merck), HCl 30% (Suprapur, Merck) and double-deionized water (resistivity 18.2 MΩ·cm) dispensed via a purification system (PureLab, Elga).

For each wine, a 400 μL aliquot is taken and diluted tenfold by 1% nitric acid in a 15×45 mm (diameter×height) Wheatstone vial made of polypropylene and is then placed in an Agilent integrated automatic sample sampler (I-AS).

The analysis is performed in semi-quantitative mode by virtue of an ICP-MS Agilent® 7700 appliance. Said appliance is equipped with an octopole that will be used in helium collision mode (He flow rate=4.3 mL/min) before being introduced into the quadrupole mass analyzer. Each sample is then nebulized by means of a Micromist® nebulizer and then introduced into the ICP-MS appliance after passing through a Scott chamber, cooled to 2° C. A 60 second duration for balancing and stabilizing the signal is programmed before proceeding to the actual measurement.

The analysis is then performed over the entire range of masses (7 to 238) at a rate of 6 acquisition points per mass and 0.1 second per point.

The semi-quantitative data is obtained by calibrated use at a point using a multi-elementary standard (5 ppb/element). This was prepared from monoelementary solutions of 1,000 ppm of Li, Mg, Co, Y, Ce, Tl (Plasma CAL ICP-MS) diluted in 1% nitric acid.

After each analysis, the needle of the sampler is rinsed in water for 30 seconds with 2% HCl and, finally, for 30 seconds with 1% HNO₃.

Between each sample, a white sample (1% HNO₃) is injected in order to ensure the absence of cross contamination.

The results from the 24 measured wines are presented in Tables 1 to 40 (see below). Reference solvent measurements are regularly performed between different analyses in order to observe the relevance of the obtained signals. For the various studied elements, several mineral elements emerge and yield usable measurements for certain samples that are representative for integrating into a metallic profile file.

Thus, 60 elements are found that can be directly integrated into a metallic profile file of the wines: Li, B, Na, Mg, Al, Si, P, S, K, Ca, Ti, V, Cr, Mn, Fe, Co, Ni, Cu, Zn, Ga, Ge, As, Br, Rb, Sr, Y, Zr, Nb, Mo, Pd, Cd, In, Sn, Sb, I, Cs, Ba, La, Ce, Pr, Nd, Sm, Eu, Gd, Tb, Dy, Ho, Er, Tm, Yb, Lu, Hf, W, Pt, Hg, Tl, Pb, Bi, Th and U.

TABLE 1

| Elements | | 7 Li [1] | | 11 B [1] | | 23 Na [1] | |
|---|---|---|---|---|---|---|---|
| | Sample | | Sequence | | Sequence | | Sequence |
| Type | name | Conc. | Unit | Conc. | Unit | Conc. | Unit |
| SQBlk | White HNO3 | <103.552 | ng/l | 398.8816858 | ng/l | 45.45254941 | ug/l |
| SQStd | Standard 5 ppb | 5 | ug/l | 750.0810035 | ng/l | 13.0744517 | ug/l |
| Sample | white | <213.659 | ng/l | 654.3456327 | ng/l | 171.0448974 | ug/l |
| Sample | Sample 2 | <213.659 | ng/l | 359.9782038 | ug/l | 10.05242045 | mg/l |
| Sample | Sample 4 | 384.6041621 | ng/l | 742.4256963 | ug/l | 15.98488748 | mg/l |
| Sample | Sample 6 | 470.0656679 | ng/l | 402.5786677 | ug/l | 7.555332725 | mg/l |
| Sample | Sample 8 | 811.9413592 | ng/l | 920.9534152 | ug/l | 16.2781122 | mg/l |
| Sample | Sample 10 | 213.6674575 | ng/l | 358.7295705 | ug/l | 8.909715839 | mg/l |
| Sample | Sample 12 | 299.1312455 | ng/l | 580.485693 | ug/l | 5.829459506 | mg/l |
| Sample | Sample 14 | 769.2083241 | ng/l | 465.4448197 | ug/l | 16.53970062 | mg/l |
| Sample | Sample 16 | 940.1541572 | ng/l | 852.2690969 | ug/l | 3.303454617 | mg/l |
| Sample | Sample 18 | 341.8665627 | ng/l | 734.920246 | ug/l | 6.439206101 | mg/l |
| Sample | Sample 36 | 213.6697396 | ng/l | 526.8016164 | ug/l | 10.28684824 | mg/l |
| Sample | Sample 20 | 384.6018799 | ng/l | 796.3726455 | ug/l | 5.25098608 | mg/l |
| Sample | Sample 22 | 726.4775712 | ng/l | 619.9668411 | ug/l | 3.48944705 | mg/l |
| Sample | Sample 24 | 683.7445361 | ng/l | 746.4599472 | ug/l | 4.604694098 | mg/l |
| Sample | Sample 26 | <213.659 | ng/l | 738.869366 | ug/l | 3.580611378 | mg/l |
| Sample | Sample 28 | 512.8055494 | ng/l | 654.659762 | ug/l | 12.76432353 | mg/l |
| Sample | Sample 30 | 299.1358098 | ng/l | 955.5213278 | ug/l | 2.791173356 | mg/l |
| Sample | Sample 32 | 256.4004926 | ng/l | 1.037840427 | mg/l | 5.334788826 | mg/l |
| Sample | Sample 34 | 1.666627164 | ug/l | 912.6039489 | ug/l | 8.948797111 | mg/l |
| Sample | Sample 38 | 470.0679501 | ng/l | 1.005119495 | mg/l | 6.078678902 | mg/l |
| Sample | Sample 40 | 256.4004926 | ng/l | 794.7387494 | ug/l | 3.15450308 | mg/l |
| Sample | Sample 42 | 2.350378547 | ug/l | 1.079250882 | mg/l | 4.292583188 | mg/l |
| Sample | Sample 44 | <213.659 | ng/l | 562.6210608 | ug/l | 15.06651499 | mg/l |
| Sample | Sample 46 | 299.140374 | ng/l | 518.9271253 | ug/l | 5.482993173 | mg/l |
| Sample | Sample 48 | 598.2716196 | ng/l | 631.7967634 | ug/l | 4.14223899 | mg/l |

TABLE 2

| Elements | | 24 Mg [1] | | 27 Al [1] | | 28 Si [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 1.345842545 | ug/l | 7.40548681 | ug/l | 9.051290487 | mg/l |
| SQStd | Standard 5 ppb | 3.654157455 | ug/l | 616.7442581 | ng/l | 16.35963502 | mg/l |
| Sample | white | 10.07497089 | ug/l | 37.4557414 | ug/l | 6.361892695 | mg/l |
| Sample | Sample 2 | 40.21929962 | mg/l | 94.81724364 | ug/l | 27.39783213 | mg/l |
| Sample | Sample 4 | 57.2107198 | mg/l | 219.4351733 | ug/l | 17.82609081 | mg/l |
| Sample | Sample 6 | 63.37641549 | mg/l | 227.125556 | ug/l | 17.35540313 | mg/l |
| Sample | Sample 8 | 107.318484 | mg/l | 361.823283 | ug/l | 34.28625214 | mg/l |
| Sample | Sample 10 | 53.98576093 | mg/l | 262.8644824 | ug/l | 8.543965339 | mg/l |
| Sample | Sample 12 | 67.89214409 | mg/l | 246.0741832 | ug/l | 10.13551967 | mg/l |
| Sample | Sample 14 | 71.58987822 | mg/l | 195.7904399 | ug/l | 17.67244989 | mg/l |
| Sample | Sample 16 | 81.64827711 | mg/l | 138.90146 | ug/l | 22.71279172 | mg/l |
| Sample | Sample 18 | 74.35237903 | mg/l | 265.0362902 | ug/l | 10.9154815 | mg/l |
| Sample | Sample 36 | 56.07988507 | mg/l | 123.8335177 | ug/l | 5.044348629 | mg/l |
| Sample | Sample 20 | 70.93663343 | mg/l | 637.9818287 | ug/l | 11.94538654 | mg/l |
| Sample | Sample 22 | 63.72679332 | mg/l | 167.3999121 | ug/l | 9.645412092 | mg/l |
| Sample | Sample 24 | 60.47740599 | mg/l | 224.5402625 | ug/l | 6.963168192 | mg/l |
| Sample | Sample 26 | 71.6240248 | mg/l | 119.6713578 | ug/l | 6.893442303 | mg/l |
| Sample | Sample 28 | 51.74325409 | mg/l | 401.8371394 | ug/l | 9.300564458 | mg/l |
| Sample | Sample 30 | 105.2148044 | mg/l | 98.68634273 | ug/l | 15.17069421 | mg/l |
| Sample | Sample 32 | 85.70104104 | mg/l | 221.7817613 | ug/l | 16.50040107 | mg/l |
| Sample | Sample 34 | 91.56334466 | mg/l | 289.0451644 | ug/l | 23.55377182 | mg/l |
| Sample | Sample 38 | 80.1787167 | mg/l | 488.871324 | ug/l | 16.87641962 | mg/l |
| Sample | Sample 40 | 78.14063082 | mg/l | 191.8597382 | ug/l | 18.06573969 | mg/l |
| Sample | Sample 42 | 77.09866078 | mg/l | 128.0541569 | ug/l | 11.32214 | mg/l |
| Sample | Sample 44 | 67.34456364 | mg/l | 223.6006067 | ug/l | 8.66382848 | mg/l |
| Sample | Sample 46 | 74.94590983 | mg/l | 549.2934725 | ug/l | 10.9343983 | mg/l |
| Sample | Sample 48 | 86.5180001 | mg/l | 44.01638581 | ug/l | 31.71849665 | mg/l |

TABLE 3

| Elements | | 31 P [1] | | 34 S [1] | | 35 Cl [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 571.5307532 | ug/l | 4.02570353 | mg/l | 47.97282426 | mg/l |
| SQStd | Standard 5 ppb | 805.2406943 | ug/l | 3.822329819 | mg/l | 131.9479021 | mg/l |
| Sample | white | 651.5369963 | ug/l | 3.869219713 | mg/l | 165.4214632 | mg/l |
| Sample | Sample 2 | 102.4006693 | mg/l | 31.9145371 | mg/l | 241.1856513 | mg/l |
| Sample | Sample 4 | 159.1210017 | mg/l | 34.39216757 | mg/l | 369.5097711 | mg/l |
| Sample | Sample 6 | 280.0174578 | mg/l | 49.96239704 | mg/l | 395.2163338 | mg/l |
| Sample | Sample 8 | 517.8048289 | mg/l | 133.9802289 | mg/l | 431.8998543 | mg/l |
| Sample | Sample 10 | 180.5712423 | mg/l | 34.39961322 | mg/l | 416.650337 | mg/l |
| Sample | Sample 12 | 185.0426363 | mg/l | 40.16886455 | mg/l | 451.3138909 | mg/l |
| Sample | Sample 14 | 261.171711 | mg/l | 47.27870729 | mg/l | 466.832573 | mg/l |
| Sample | Sample 16 | 308.7028858 | mg/l | 55.12642359 | mg/l | 503.9312652 | mg/l |
| Sample | Sample 18 | 308.2967901 | mg/l | 55.49597322 | mg/l | 491.2374341 | mg/l |
| Sample | Sample 36 | 168.2671848 | mg/l | 70.80481424 | mg/l | 504.764169 | mg/l |
| Sample | Sample 20 | 308.8737106 | mg/l | 58.10062739 | mg/l | 502.2557072 | mg/l |
| Sample | Sample 22 | 220.3018818 | mg/l | 40.88789194 | mg/l | 514.4503573 | mg/l |
| Sample | Sample 24 | 259.1332598 | mg/l | 51.75000478 | mg/l | 482.013051 | mg/l |
| Sample | Sample 26 | 268.8340551 | mg/l | 57.56222708 | mg/l | 499.7608859 | mg/l |
| Sample | Sample 28 | 236.4148899 | mg/l | 56.12194316 | mg/l | 500.8959476 | mg/l |
| Sample | Sample 30 | 297.9603752 | mg/l | 73.22919105 | mg/l | 503.781959 | mg/l |
| Sample | Sample 32 | 273.0902129 | mg/l | 55.53016172 | mg/l | 538.3748978 | mg/l |
| Sample | Sample 34 | 277.3785465 | mg/l | 65.36213504 | mg/l | 579.9381989 | mg/l |
| Sample | Sample 38 | 300.9018453 | mg/l | 66.56772107 | mg/l | 559.1871039 | mg/l |
| Sample | Sample 40 | 282.9169638 | mg/l | 53.16221228 | mg/l | 551.7677889 | mg/l |
| Sample | Sample 42 | 354.0737902 | mg/l | 70.31715412 | mg/l | 589.9467349 | mg/l |
| Sample | Sample 44 | 144.9746257 | mg/l | 57.02404249 | mg/l | 551.5583073 | mg/l |
| Sample | Sample 46 | 202.4527129 | mg/l | 52.44314893 | mg/l | 531.898792 | mg/l |
| Sample | Sample 48 | 251.9763014 | mg/l | 65.34264171 | mg/l | 579.0035088 | mg/l |

TABLE 4

| Elements | | 39 K [1] | | 42 Ca [1] | | 47 Ti [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 109.9271848 | ug/l | 165.3536709 | ug/l | 311.1896826 | ng/l |
| SQStd | Standard 5 ppb | −73821.17613 | ng/l | 45.35820695 | ug/l | −73.45879846 | ng/l |
| Sample | white | 42.8633215 | ug/l | 55.57808019 | ug/l | −73.45504155 | ng/l |
| Sample | Sample 2 | 109.223088 | mg/l | 2.336192958 | mg/l | 15.95873514 | ug/l |
| Sample | Sample 4 | 70.36323798 | mg/l | 3.438258973 | mg/l | 14.56632152 | ug/l |
| Sample | Sample 6 | 210.7687553 | mg/l | 2.439314046 | mg/l | 11.81450218 | ug/l |
| Sample | Sample 8 | 294.0839828 | mg/l | 5.701556988 | mg/l | 31.96113103 | ug/l |
| Sample | Sample 10 | 110.4991892 | mg/l | 2.789134066 | mg/l | 6.549551335 | ug/l |
| Sample | Sample 12 | 230.7014843 | mg/l | 4.440908287 | mg/l | 7.772323343 | ug/l |
| Sample | Sample 14 | 172.4592944 | mg/l | 3.187409008 | mg/l | 8.893114297 | ug/l |
| Sample | Sample 16 | 259.4974877 | mg/l | 3.197875336 | mg/l | 6.685380288 | ug/l |
| Sample | Sample 18 | 226.6419925 | mg/l | 3.546186186 | mg/l | 16.91005082 | ug/l |
| Sample | Sample 36 | 219.4981235 | mg/l | 3.435322498 | mg/l | 5.326729581 | ug/l |
| Sample | Sample 20 | 205.5121101 | mg/l | 3.257873386 | mg/l | 63.09468983 | ug/l |
| Sample | Sample 22 | 179.8242421 | mg/l | 2.821637882 | mg/l | 18.84641141 | ug/l |
| Sample | Sample 24 | 204.7360943 | mg/l | 3.353109128 | mg/l | 7.262769412 | ug/l |
| Sample | Sample 26 | 202.5210048 | mg/l | 3.690330032 | mg/l | 6.107916026 | ug/l |
| Sample | Sample 28 | 184.0355298 | mg/l | 3.825825088 | mg/l | 5.768228605 | ug/l |
| Sample | Sample 30 | 307.7550055 | mg/l | 3.289413397 | mg/l | 8.28178659 | ug/l |
| Sample | Sample 32 | 384.2718882 | mg/l | 2.694392437 | mg/l | 11.81435191 | ug/l |
| Sample | Sample 34 | 266.7026193 | mg/l | 3.296355406 | mg/l | 8.859134258 | ug/l |
| Sample | Sample 38 | 289.7568031 | mg/l | 3.916694127 | mg/l | 36.4467573 | ug/l |
| Sample | Sample 40 | 269.9992903 | mg/l | 3.685556756 | mg/l | 7.908209815 | ug/l |
| Sample | Sample 42 | 343.482673 | mg/l | 3.371464758 | mg/l | 11.13503925 | ug/l |
| Sample | Sample 44 | 161.4958978 | mg/l | 3.330115058 | mg/l | 4.61344984 | ug/l |
| Sample | Sample 46 | 143.4423814 | mg/l | 4.084423327 | mg/l | 35.35889445 | ug/l |
| Sample | Sample 48 | 247.8288518 | mg/l | 2.92514439 | mg/l | 7.092993585 | ug/l |

TABLE 5

| Elements | | 51 V [1] | | 52 Cr [1] | | 55 Mn [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 16.81311052 | ng/l | 67.67902381 | ng/l | 155.3287517 | ng/l |
| SQStd | Standard 5 ppb | 31.86257881 | ng/l | −37.06057744 | ng/l | −110.1310209 | ng/l |
| Sample | white | 22.41714202 | ng/l | −30.10183061 | ng/l | −31.03014683 | ng/l |
| Sample | Sample 2 | 102.3342998 | ng/l | 1.287569019 | ug/l | 64.13055815 | ug/l |
| Sample | Sample 4 | 1.438390426 | ug/l | 1.955104395 | ug/l | 90.61750099 | ug/l |
| Sample | Sample 6 | 249.1038201 | ng/l | 6.18275747 | ug/l | 189.962924 | ug/l |
| Sample | Sample 8 | 4.244353229 | ug/l | 5.542214544 | ug/l | 390.3501441 | ug/l |
| Sample | Sample 10 | 435.1334524 | ng/l | 917.7918427 | ng/l | 85.60138964 | ug/l |
| Sample | Sample 12 | 509.9894998 | ng/l | 3.579203746 | ug/l | 155.041045 | ug/l |
| Sample | Sample 14 | 254.1882554 | ng/l | 1.945328556 | ug/l | 187.1458109 | ug/l |
| Sample | Sample 16 | 159.0060282 | ng/l | 1.745393412 | ug/l | 127.3844659 | ug/l |
| Sample | Sample 18 | 14.88975118 | ug/l | 3.499016227 | ug/l | 117.1293001 | ug/l |
| Sample | Sample 36 | 267.961403 | ng/l | 1.854222695 | ug/l | 109.1439213 | ug/l |
| Sample | Sample 20 | 52.66518139 | ug/l | 6.551584221 | ug/l | 128.2843595 | ug/l |
| Sample | Sample 22 | 20.6926209 | ug/l | 4.225234043 | ug/l | 183.3240207 | ug/l |
| Sample | Sample 24 | 183.7117237 | ng/l | 1.551070106 | ug/l | 120.9878321 | ug/l |
| Sample | Sample 26 | 141.567232 | ng/l | 1.705412114 | ug/l | 136.0095237 | ug/l |
| Sample | Sample 28 | 440.9521153 | ng/l | 2.270441618 | ug/l | 76.04574577 | ug/l |
| Sample | Sample 30 | 68.91401133 | ng/l | 1.558969556 | ug/l | 139.7247233 | ug/l |
| Sample | Sample 32 | 11.80028951 | ug/l | 2.462592264 | ug/l | 149.3847822 | ug/l |
| Sample | Sample 34 | 634.9954224 | ng/l | 2.551480163 | ug/l | 129.6985654 | ug/l |
| Sample | Sample 38 | 142.1336108 | ug/l | 5.148597449 | ug/l | 82.57292303 | ug/l |
| Sample | Sample 40 | 329.0302474 | ng/l | 2.079709815 | ug/l | 118.0014867 | ug/l |
| Sample | Sample 42 | 249.83102 | ng/l | 1.402812882 | ug/l | 67.8505192 | ug/l |
| Sample | Sample 44 | 1.322770396 | ug/l | 3.005367289 | ug/l | 139.0684285 | ug/l |
| Sample | Sample 46 | 55.9035418 | ug/l | 4.264372733 | ug/l | 120.6679044 | ug/l |
| Sample | Sample 48 | 95.79650674 | ng/l | 2.14903218 | ug/l | 255.1467676 | ug/l |

TABLE 6

| Elements | | 56 Fe [1] | | 59 Co [1] | | 60 Ni [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 697.769771 | ng/l | 58.50541754 | ng/l | 980.0737576 | ng/l |
| SQStd | Standard 5 ppb | −363.6331141 | ng/l | 4.941494582 | ug/l | −217.2142673 | ng/l |
| Sample | white | 140.9956665 | ng/l | −16.65157149 | ng/l | −282.2048182 | ng/l |
| Sample | Sample 2 | 47.91086128 | ug/l | 143.8044491 | ng/l | 310.8375716 | ng/l |
| Sample | Sample 4 | 54.25910377 | ug/l | 135.4290357 | ng/l | 656.4293359 | ng/l |
| Sample | Sample 6 | 357.2513567 | ug/l | 468.2552451 | ng/l | 3.478566007 | ug/l |
| Sample | Sample 8 | 363.1949562 | ug/l | 756.1226898 | ng/l | 2.789697908 | ug/l |
| Sample | Sample 10 | 141.8671054 | ug/l | 203.7987966 | ng/l | 1.300366413 | ug/l |
| Sample | Sample 12 | 463.0314151 | ug/l | 366.8931267 | ng/l | 3.972228923 | ug/l |
| Sample | Sample 14 | 106.794416 | ug/l | 332.5923871 | ng/l | 1.467770227 | ug/l |
| Sample | Sample 16 | 338.6891301 | ug/l | 177.8923604 | ng/l | 918.0470464 | ng/l |
| Sample | Sample 18 | 241.3732767 | ug/l | 263.8047633 | ng/l | 1.500704344 | ug/l |
| Sample | Sample 36 | 419.7089698 | ug/l | 140.4935989 | ng/l | 918.0636158 | ng/l |
| Sample | Sample 20 | 353.9049141 | ug/l | 353.6396037 | ng/l | 3.877025143 | ug/l |
| Sample | Sample 22 | 251.4645228 | ug/l | 283.680842 | ng/l | 1.333238529 | ug/l |
| Sample | Sample 24 | 300.3020631 | ug/l | 150.8158555 | ng/l | 481.0532713 | ng/l |
| Sample | Sample 26 | 269.9888019 | ug/l | 132.5086044 | ng/l | 1.767619072 | ug/l |
| Sample | Sample 28 | 46.81653605 | ug/l | 186.0730751 | ng/l | 2.660843075 | ug/l |
| Sample | Sample 30 | 133.7489445 | ug/l | 98.2297607 | ng/l | 492.7497897 | ng/l |
| Sample | Sample 32 | 221.9995518 | ug/l | 274.7157015 | ng/l | 687.8387192 | ng/l |
| Sample | Sample 34 | 486.0647215 | ug/l | 252.3121766 | ng/l | 1.425334937 | ug/l |
| Sample | Sample 38 | 360.4894979 | ug/l | 177.3068389 | ng/l | 994.0870657 | ng/l |
| Sample | Sample 40 | 285.565494 | ug/l | 122.1848271 | ng/l | 1.037203123 | ug/l |
| Sample | Sample 42 | 189.4034673 | ug/l | 100.957121 | ng/l | 655.6712447 | ng/l |
| Sample | Sample 44 | 199.1401473 | ug/l | 482.8690444 | ng/l | 1.274753087 | ug/l |
| Sample | Sample 46 | 339.5462704 | ug/l | 256.0169148 | ng/l | 7.869439724 | ug/l |
| Sample | Sample 48 | 137.5565211 | ug/l | 377.2240324 | ng/l | 6.835329243 | ug/l |

TABLE 7

| Elements | | 63 Cu [1] | | 66 Zn [1] | | 69 Ga [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 378.5327515 | ng/l | 3.87542571 | ug/l | 26.62986063 | ng/l |
| SQStd | Standard 5 ppb | −46.85820073 | ng/l | −2956.246044 | ng/l | −5.999480874 | ng/l |
| Sample | white | −68.59945931 | ng/l | −2370.045563 | ng/l | 101.0260788 | ng/l |
| Sample | Sample 2 | 2.36263051 | ug/l | 105.4973343 | ug/l | 1.51134778 | ug/l |
| Sample | Sample 4 | 919.7228643 | ng/l | 96.45065504 | ug/l | 1.82384307 | ug/l |
| Sample | Sample 6 | 1.034387006 | ug/l | 191.7477882 | ug/l | 7.158059772 | ug/l |
| Sample | Sample 8 | 17.67211005 | ug/l | 230.3661671 | ug/l | 9.098203663 | ug/l |
| Sample | Sample 10 | 9.79786708 | ug/l | 86.0609195 | ug/l | 1.573976054 | ug/l |
| Sample | Sample 12 | 9.426082494 | ug/l | 100.1904827 | ug/l | 4.417525022 | ug/l |
| Sample | Sample 14 | 1.281366681 | ug/l | 165.7214305 | ug/l | 4.216881798 | ug/l |
| Sample | Sample 16 | 8.074208412 | ug/l | 134.241582 | ug/l | 3.551889301 | ug/l |
| Sample | Sample 18 | 10.33128007 | ug/l | 100.5224185 | ug/l | 2.0098397 | ug/l |
| Sample | Sample 36 | 7.666715068 | ug/l | 121.5925046 | ug/l | 1.721171487 | ug/l |
| Sample | Sample 20 | 6.173876224 | ug/l | 67.77254384 | ug/l | 2.832944584 | ug/l |
| Sample | Sample 22 | 12.75528479 | ug/l | 114.4558679 | ug/l | 1.65338826 | ug/l |
| Sample | Sample 24 | 11.99647668 | ug/l | 35.31989163 | ug/l | 1.428074041 | ug/l |
| Sample | Sample 26 | 12.33598496 | ug/l | 98.63813844 | ug/l | 1.080845186 | ug/l |
| Sample | Sample 28 | 8.44007987 | ug/l | 187.8174597 | ug/l | 4.017014185 | ug/l |
| Sample | Sample 30 | 3.802327859 | ug/l | 73.97119911 | ug/l | 1.351283615 | ug/l |
| Sample | Sample 32 | 23.49983002 | ug/l | 296.9762928 | ug/l | 1.675984558 | ug/l |
| Sample | Sample 34 | 10.72818774 | ug/l | 132.0289083 | ug/l | 2.477520892 | ug/l |
| Sample | Sample 38 | 6.758202201 | ug/l | 87.9489938 | ug/l | 4.367734787 | ug/l |
| Sample | Sample 40 | 9.327647031 | ug/l | 143.0941842 | ug/l | 5.059090523 | ug/l |
| Sample | Sample 42 | 5.444868565 | ug/l | 59.78116687 | ug/l | 1.612691002 | ug/l |
| Sample | Sample 44 | 4.272982327 | ug/l | 73.36907034 | ug/l | 1.687609761 | ug/l |
| Sample | Sample 46 | 107.6118743 | ug/l | 152.3533326 | ug/l | 2.562828093 | ug/l |
| Sample | Sample 48 | 1.423896874 | ug/l | 115.9017017 | ug/l | 4.442089308 | ug/l |

TABLE 8

| Elements | | 72 Ge [1] | | 75 As [1] | | 79 Br [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | <26.255 | ng/l | 71.71071852 | ng/l | 352.2958153 | ng/l |
| SQStd | Standard 5 ppb | <16.908 | ng/l | 213.7563353 | ng/l | 323.5535155 | ng/l |
| Sample | white | <16.908 | ng/l | −26.20223692 | ng/l | 371.8278856 | ng/l |
| Sample | Sample 2 | 253.2810792 | ng/l | 325.4666216 | ng/l | 2.930529297 | ug/l |
| Sample | Sample 4 | 111.2435368 | ng/l | 776.4496127 | ng/l | 14.59150843 | ug/l |
| Sample | Sample 6 | 253.2814662 | ng/l | 304.7763601 | ng/l | 13.022078 | ug/l |
| Sample | Sample 8 | 226.2266434 | ng/l | 1.624731522 | ug/l | 42.17505396 | ug/l |
| Sample | Sample 10 | 53.75160291 | ng/l | 271.6776356 | ng/l | 5.634214073 | ug/l |
| Sample | Sample 12 | 134.9170195 | ng/l | 486.8237637 | ng/l | 19.37266133 | ug/l |
| Sample | Sample 14 | 67.27892402 | ng/l | 615.0875153 | ng/l | 7.396482338 | ug/l |
| Sample | Sample 16 | 134.9154844 | ng/l | 329.6007033 | ng/l | 18.98612312 | ug/l |
| Sample | Sample 18 | 87.57027333 | ng/l | 2.216505813 | ug/l | 14.54324732 | ug/l |
| Sample | Sample 36 | 107.8595264 | ng/l | 155.8326364 | ng/l | 17.19921127 | ug/l |
| Sample | Sample 20 | 239.7538097 | ng/l | 9.416876896 | ug/l | 8.362183351 | ug/l |
| Sample | Sample 22 | 70.65989645 | ng/l | 449.5914624 | ng/l | 9.183080405 | ug/l |
| Sample | Sample 24 | 40.2239206 | ng/l | 201.3450948 | ng/l | 9.545294337 | ug/l |
| Sample | Sample 26 | 67.28007211 | ng/l | 321.3314983 | ng/l | 7.034487171 | ug/l |
| Sample | Sample 28 | 9.788136632 | ng/l | 255.1347752 | ng/l | 13.52912462 | ug/l |
| Sample | Sample 30 | 33.46121464 | ng/l | 52.4044563 | ng/l | 26.69057353 | ug/l |
| Sample | Sample 32 | 46.9875747 | ng/l | 1.202647209 | ug/l | 20.91815714 | ug/l |
| Sample | Sample 34 | 87.57084738 | ng/l | 590.2696424 | ng/l | 16.04028903 | ug/l |
| Sample | Sample 38 | 97.71603506 | ng/l | 10.26195477 | ug/l | 12.65971012 | ug/l |
| Sample | Sample 40 | 114.6265925 | ng/l | 404.078783 | ng/l | 12.22529235 | ug/l |
| Sample | Sample 42 | 57.13371698 | ng/l | 288.2297122 | ng/l | 12.39426369 | ug/l |
| Sample | Sample 44 | 43.60698927 | ng/l | 1.583322215 | ug/l | 7.951793014 | ug/l |
| Sample | Sample 46 | 97.71469347 | ng/l | 3.681624307 | ug/l | 5.4170306 | ug/l |
| Sample | Sample 48 | 16.5514118 | ng/l | 60.67720423 | ng/l | 16.78904338 | ug/l |

TABLE 9

| Elements | | 85 Rb [1] | | 88 Sr [1] | | 89 Y [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 77.68554883 | ng/l | 113.5781214 | ng/l | 18.37497293 | ng/l |
| SQStd | Standard 5 ppb | −73.02728374 | ng/l | −90.53121563 | ng/l | 4.981625027 | ug/l |
| Sample | white | −19.12234793 | ng/l | −40.51287492 | ng/l | −8.108227662 | ng/l |
| Sample | Sample 2 | 73.5228414 | ug/l | 11.1332065 | ug/l | 53.04173759 | ng/l |
| Sample | Sample 4 | 56.04541135 | ug/l | 47.79370159 | ug/l | 768.1358313 | ng/l |
| Sample | Sample 6 | 139.9413354 | ug/l | 51.88424904 | ug/l | 45.05434229 | ng/l |
| Sample | Sample 8 | 187.1856522 | ug/l | 79.68268141 | ug/l | 92.29121639 | ng/l |
| Sample | Sample 10 | 89.26818296 | ug/l | 19.64865762 | ug/l | 37.75230732 | ng/l |
| Sample | Sample 12 | 170.3373872 | ug/l | 38.86337096 | ug/l | 19.95533588 | ng/l |
| Sample | Sample 14 | 118.171513 | ug/l | 34.86956026 | ug/l | 102.3321167 | ng/l |
| Sample | Sample 16 | 138.4917947 | ug/l | 67.69798172 | ug/l | −15.18093225 | ng/l |
| Sample | Sample 18 | 184.4601263 | ug/l | 28.64197651 | ug/l | 42.77266001 | ng/l |
| Sample | Sample 36 | 179.3862346 | ug/l | 27.79547538 | ug/l | −9.020992476 | ng/l |
| Sample | Sample 20 | 133.5813522 | ug/l | 23.62683713 | ug/l | 513.3333115 | ng/l |
| Sample | Sample 22 | 195.8535964 | ug/l | 42.55728356 | ug/l | 51.89984168 | ng/l |
| Sample | Sample 24 | 102.7423864 | ug/l | 13.08190341 | ug/l | 3.299550208 | ng/l |
| Sample | Sample 26 | 94.70598827 | ug/l | 12.72687384 | ug/l | −2.632620439 | ng/l |
| Sample | Sample 28 | 102.7084592 | ug/l | 42.89622801 | ug/l | 15.84810308 | ng/l |
| Sample | Sample 30 | 178.6661431 | ug/l | 16.8962495 | ug/l | −9.249016589 | ng/l |
| Sample | Sample 32 | 134.411252 | ug/l | 65.04698955 | ug/l | 12.42563883 | ng/l |
| Sample | Sample 34 | 157.4400284 | ug/l | 107.306449 | ug/l | −11.75872342 | ng/l |
| Sample | Sample 38 | 99.50652884 | ug/l | 61.37992634 | ug/l | 115.8007446 | ng/l |
| Sample | Sample 40 | 149.642472 | ug/l | 78.31357277 | ug/l | −7.652021049 | ng/l |
| Sample | Sample 42 | 241.2636533 | ug/l | 132.9713789 | ug/l | 18.81488462 | ng/l |
| Sample | Sample 44 | 97.0117034 | ug/l | 22.13103182 | ug/l | 271.2438879 | ng/l |
| Sample | Sample 46 | 101.4672227 | ug/l | 29.58482174 | ug/l | 376.0573656 | ng/l |
| Sample | Sample 48 | 142.2662132 | ug/l | 45.34574052 | ug/l | −12.67129155 | ng/l |

TABLE 10

| Elements | | 90 Zr [1] | | 93 Nb [1] | | 95 Mo [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 8.978906162 | ng/l | <1.924 | ng/l | 16.51879553 | ng/l |
| SQStd | Standard 5 ppb | 12.30438249 | ng/l | <1.111 | ng/l | <4.744 | ng/l |
| Sample | white | 4.920162511 | ng/l | <1.111 | ng/l | <4.744 | ng/l |
| Sample | Sample 2 | 510.6722766 | ng/l | 127.892965 | ng/l | 215.0043653 | ng/l |
| Sample | Sample 4 | 1.337361361 | ug/l | 55.88943829 | ng/l | 332.6755589 | ng/l |
| Sample | Sample 6 | 128.2845428 | ng/l | 1.230051549 | ng/l | 70.77309754 | ng/l |
| Sample | Sample 8 | 126.1134225 | ng/l | 62.33410693 | ng/l | 502.5626329 | ng/l |
| Sample | Sample 10 | 351.6117496 | ng/l | 9.228517505 | ng/l | 79.31383509 | ng/l |
| Sample | Sample 12 | 48.79002633 | ng/l | 9.2285192 | ng/l | 207.4112394 | ng/l |
| Sample | Sample 14 | 1.609359244 | ug/l | 254.8182518 | ng/l | 459.8457422 | ng/l |
| Sample | Sample 16 | 19.25344795 | ng/l | 1.896572157 | ng/l | 217.853266 | ng/l |
| Sample | Sample 18 | 214.3024802 | ng/l | 72.77900861 | ng/l | 1.55922975 | ug/l |
| Sample | Sample 36 | 31.1576884 | ng/l | 8.339808953 | ng/l | 174.2006926 | ng/l |
| Sample | Sample 20 | 1.04198339 | ug/l | 591.3394516 | ng/l | 2.333427621 | ug/l |
| Sample | Sample 22 | 99.6136311 | ng/l | 76.11029591 | ng/l | 3.206890982 | ug/l |
| Sample | Sample 24 | 45.31561289 | ng/l | 1.007878013 | ng/l | 193.1803008 | ng/l |
| Sample | Sample 26 | 71.8126043 | ng/l | 1.674385909 | ng/l | 201.7189392 | ng/l |
| Sample | Sample 28 | 1.091568234 | ug/l | 12.78350172 | ng/l | 106.8292949 | ng/l |
| Sample | Sample 30 | 19.25374618 | ng/l | 2.340919229 | ng/l | 227.3401819 | ng/l |
| Sample | Sample 32 | 127.8489907 | ng/l | 103.8913634 | ng/l | 1.406332137 | ug/l |
| Sample | Sample 34 | 97.87614107 | ng/l | 9.450843167 | ng/l | 425.6763354 | ng/l |
| Sample | Sample 38 | 1.756500134 | ug/l | 549.5266772 | ng/l | 14.70725445 | ug/l |
| Sample | Sample 40 | 67.90296013 | ng/l | 9.450704177 | ng/l | 328.8806536 | ng/l |
| Sample | Sample 42 | 94.40148574 | ng/l | 2.118783831 | ng/l | 224.4946109 | ng/l |
| Sample | Sample 44 | 839.3259495 | ng/l | 4.56278214 | ng/l | 236.8287193 | ng/l |
| Sample | Sample 46 | 4.54175837 | ug/l | 421.1493032 | ng/l | 5.790860076 | ug/l |
| Sample | Sample 48 | 27.07229559 | ng/l | 2.340957367 | ng/l | 117.2689473 | ng/l |

TABLE 11

| Elements | | 105 Pd [1] | | 111 Cd [1] | | 115 In [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | <3.250 | ng/l | <9.153 | ng/l | <1.571 | ng/l |
| SQStd | Standard 5 ppb | 111.921373 | ng/l | <5.097 | ng/l | <0.870 | ng/l |
| Sample | white | <1.829 | ng/l | 0.624382298 | ng/l | <0.870 | ng/l |
| Sample | Sample 2 | 12.43465639 | ng/l | 6.740748179 | ng/l | 0.870462046 | ng/l |
| Sample | Sample 4 | 35.11022088 | ng/l | −0.394873164 | ng/l | 1.044562524 | ng/l |
| Sample | Sample 6 | 16.09183918 | ng/l | 16.93454906 | ng/l | 0.870451836 | ng/l |
| Sample | Sample 8 | 32.5502073 | ng/l | 42.41958107 | ng/l | 2.089104793 | ng/l |
| Sample | Sample 10 | 4.388639499 | ng/l | 122.956231 | ng/l | 1.392733098 | ng/l |
| Sample | Sample 12 | 12.80028692 | ng/l | 27.1290071 | ng/l | 2.785507368 | ng/l |
| Sample | Sample 14 | 8.045910521 | ng/l | 12.85752721 | ng/l | 2.95958701 | ng/l |
| Sample | Sample 16 | 13.89767515 | ng/l | 5.721372173 | ng/l | <0.870 | ng/l |
| Sample | Sample 18 | 6.948697372 | ng/l | 17.95439946 | ng/l | 1.915075621 | ng/l |
| Sample | Sample 36 | 8.411689624 | ng/l | 31.20681053 | ng/l | <0.870 | ng/l |
| Sample | Sample 20 | 5.120178523 | ng/l | 19.99314953 | ng/l | 1.740934219 | ng/l |
| Sample | Sample 22 | 8.777449893 | ng/l | 25.09025897 | ng/l | 1.392743391 | ng/l |
| Sample | Sample 24 | 2.560078799 | ng/l | 4.701939785 | ng/l | 1.044562524 | ng/l |
| Sample | Sample 26 | 4.023135571 | ng/l | 29.16758023 | ng/l | 2.959597636 | ng/l |
| Sample | Sample 28 | 13.53184652 | ng/l | 8.779556572 | ng/l | <0.870 | ng/l |
| Sample | Sample 30 | 4.022901899 | ng/l | 30.18755118 | ng/l | 1.566884627 | ng/l |
| Sample | Sample 32 | 16.45749482 | ng/l | 17.95458028 | ng/l | <0.870 | ng/l |
| Sample | Sample 34 | 28.16116498 | ng/l | 14.89610035 | ng/l | 1.218632537 | ng/l |
| Sample | Sample 38 | 16.45753667 | ng/l | 16.93496513 | ng/l | 4.178229508 | ng/l |
| Sample | Sample 40 | 19.74921866 | ng/l | 12.85717141 | ng/l | <0.870 | ng/l |
| Sample | Sample 42 | 28.89247068 | ng/l | 3.682684323 | ng/l | 2.785496743 | ng/l |
| Sample | Sample 44 | 2.194298824 | ng/l | 19.99302898 | ng/l | <0.870 | ng/l |
| Sample | Sample 46 | 6.948739921 | ng/l | 31.20651112 | ng/l | 18.10608909 | ng/l |
| Sample | Sample 48 | 12.43461594 | ng/l | 52.61440657 | ng/l | 2.263185265 | ng/l |

TABLE 12

| Elements | | 118 Sn [1] | | 121 Sb [1] | | 125 Te [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 19.85148751 | ng/l | 12.72601987 | ng/l | <163.334 | ng/l |
| SQStd | Standard 5 ppb | −13.69617088 | ng/l | <4.362 | ng/l | <89.138 | ng/l |
| Sample | white | −6.172880895 | ng/l | −5.746184434 | ng/l | <89.138 | ng/l |
| Sample | Sample 2 | 74.53388208 | ng/l | 51.83887793 | ng/l | <89.138 | ng/l |
| Sample | Sample 4 | 56.74967791 | ng/l | 63.18083792 | ng/l | <89.138 | ng/l |
| Sample | Sample 6 | 122.4127437 | ng/l | 41.3683639 | ng/l | <89.138 | ng/l |
| Sample | Sample 8 | 798.4001179 | ng/l | 33.51594847 | ng/l | <89.138 | ng/l |
| Sample | Sample 10 | 91.63160162 | ng/l | 167.8886474 | ng/l | <89.138 | ng/l |
| Sample | Sample 12 | 531.5142635 | ng/l | 181.8500605 | ng/l | <89.138 | ng/l |
| Sample | Sample 14 | 295.4757525 | ng/l | 120.768963 | ng/l | <89.138 | ng/l |
| Sample | Sample 16 | 197.6534517 | ng/l | 82.37780925 | ng/l | <89.138 | ng/l |
| Sample | Sample 18 | 376.8892288 | ng/l | 496.0204284 | ng/l | <89.138 | ng/l |
| Sample | Sample 36 | 142.2488779 | ng/l | 62.30838355 | ng/l | <89.138 | ng/l |
| Sample | Sample 20 | 383.0468764 | ng/l | 110.2989914 | ng/l | <89.138 | ng/l |
| Sample | Sample 22 | 257.8522626 | ng/l | 29.15356349 | ng/l | <89.138 | ng/l |
| Sample | Sample 24 | 212.702831 | ng/l | 34.38847272 | ng/l | <89.138 | ng/l |
| Sample | Sample 26 | 499.3578773 | ng/l | 235.0810789 | ng/l | <89.138 | ng/l |
| Sample | Sample 28 | 163.4518976 | ng/l | 30.02649377 | ng/l | <89.138 | ng/l |
| Sample | Sample 30 | 385.7892252 | ng/l | 5.596089223 | ng/l | <89.138 | ng/l |
| Sample | Sample 32 | 73.84868099 | ng/l | 34.38846939 | ng/l | <89.138 | ng/l |
| Sample | Sample 34 | 344.812109 | ng/l | 89.35763053 | ng/l | <89.138 | ng/l |
| Sample | Sample 38 | 1.188576753 | ug/l | 450.6354421 | ng/l | <89.138 | ng/l |
| Sample | Sample 40 | 106.6792069 | ng/l | 19.55616944 | ng/l | <89.138 | ng/l |
| Sample | Sample 42 | 578.7258726 | ng/l | 36.13299206 | ng/l | <89.138 | ng/l |
| Sample | Sample 44 | 18.44875084 | ng/l | 117.2791322 | ng/l | <89.138 | ng/l |
| Sample | Sample 46 | 4.029092664 | ug/l | 452.3793657 | ng/l | <89.138 | ng/l |
| Sample | Sample 48 | 535.6183322 | ng/l | 65.7994324 | ng/l | <89.138 | ng/l |

TABLE 13

| Elements | | 127 I [1] | | 133 Cs [1] | | 137 Ba [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 41.58646974 | ng/l | <2.032 | ng/l | 227.9071185 | ng/l |
| SQStd | Standard 5 ppb | −14.24935188 | ng/l | <1.093 | ng/l | −148.2697642 | ng/l |
| Sample | white | 7.14497212 | ng/l | <1.093 | ng/l | 422.9750692 | ng/l |
| Sample | Sample 2 | 168.7985149 | ng/l | 762.4770882 | ng/l | 5.846943887 | ug/l |
| Sample | Sample 4 | 587.2631514 | ng/l | 492.9569838 | ng/l | 6.904220065 | ug/l |
| Sample | Sample 6 | 905.9206168 | ng/l | 228.6837993 | ng/l | 27.90869193 | ug/l |
| Sample | Sample 8 | 1.712314109 | ug/l | 386.6133597 | ng/l | 34.45411196 | ug/l |
| Sample | Sample 10 | 273.4107976 | ng/l | 1.302950186 | ug/l | 5.960534584 | ug/l |
| Sample | Sample 12 | 580.1336105 | ng/l | 2.490152582 | ug/l | 17.00340791 | ug/l |
| Sample | Sample 14 | 349.4916961 | ng/l | 2.606341951 | ug/l | 16.4074193 | ug/l |
| Sample | Sample 16 | 1.046238945 | ug/l | 649.9142549 | ng/l | 12.80062686 | ug/l |
| Sample | Sample 18 | 1.100952947 | ug/l | 1.183861637 | ug/l | 7.030303083 | ug/l |
| Sample | Sample 36 | 1.091440581 | ug/l | 409.8106665 | ng/l | 6.609606138 | ug/l |
| Sample | Sample 20 | 611.0449722 | ng/l | 1.345929076 | ug/l | 9.865379646 | ug/l |
| Sample | Sample 22 | 457.6742443 | ng/l | 5.766391818 | ug/l | 6.115203766 | ug/l |
| Sample | Sample 24 | 1.845551208 | ug/l | 408.059719 | ng/l | 5.022200958 | ug/l |
| Sample | Sample 26 | 392.2862063 | ng/l | 387.0518969 | ng/l | 3.859888305 | ug/l |
| Sample | Sample 28 | 557.5363832 | ng/l | 468.4522021 | ng/l | 14.94934629 | ug/l |
| Sample | Sample 30 | 589.6429294 | ng/l | 627.3700052 | ng/l | 4.656733416 | ug/l |
| Sample | Sample 32 | 1.115215656 | ug/l | 559.932274 | ng/l | 6.295007656 | ug/l |
| Sample | Sample 34 | 960.6303387 | ng/l | 534.5443886 | ng/l | 9.420356636 | ug/l |
| Sample | Sample 38 | 788.2054641 | ng/l | 411.3410649 | ng/l | 17.13537742 | ug/l |
| Sample | Sample 40 | 738.2649462 | ng/l | 428.8446443 | ng/l | 18.57949393 | ug/l |
| Sample | Sample 42 | 829.8301792 | ng/l | 618.3823663 | ng/l | 6.389888424 | ug/l |
| Sample | Sample 44 | 253.1983536 | ng/l | 540.2344878 | ng/l | 6.828052262 | ug/l |
| Sample | Sample 46 | 416.0659597 | ng/l | 997.9808644 | ng/l | 10.20690691 | ug/l |
| Sample | Sample 48 | 2.0763756 | ug/l | 569.5652599 | ng/l | 17.26043186 | ug/l |

TABLE 14

| Elements | | 139 La [1] | | 140 Ce [1] | | 141 Pr [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 7.820335522 | ng/l | 25.57580014 | ng/l | <0.657 | ng/l |
| SQStd | Standard 5 ppb | <0.612 | ng/l | 4.9744242 | ug/l | <0.353 | ng/l |
| Sample | white | −4.391149716 | ng/l | −9.603464179 | ng/l | <0.353 | ng/l |
| Sample | Sample 2 | 13.6130943 | ng/l | 26.12190138 | ng/l | 4.764811859 | ng/l |
| Sample | Sample 4 | 22.43233101 | ng/l | 40.84028507 | ng/l | 3.987430102 | ng/l |
| Sample | Sample 6 | 41.90879096 | ng/l | 29.89866785 | ng/l | 8.440029698 | ng/l |
| Sample | Sample 8 | 377.7134986 | ng/l | 351.013893 | ng/l | 52.69080508 | ng/l |
| Sample | Sample 10 | 8.101412621 | ng/l | 14.01933895 | ng/l | 2.220604982 | ng/l |
| Sample | Sample 12 | 7.366424685 | ng/l | 1.432805414 | ng/l | 1.443232795 | ng/l |
| Sample | Sample 14 | 24.88191582 | ng/l | 42.7771561 | ng/l | 7.026441825 | ng/l |
| Sample | Sample 16 | −4.636086521 | ng/l | −21.99430929 | ng/l | <0.353 | ng/l |
| Sample | Sample 18 | 49.38117237 | ng/l | 81.32304767 | ng/l | 8.227727634 | ng/l |
| Sample | Sample 36 | −3.288891584 | ng/l | −17.73503056 | ng/l | 0.312517351 | ng/l |
| Sample | Sample 20 | 1.470335346 | ug/l | 1.885578448 | ug/l | 212.5151475 | ng/l |
| Sample | Sample 22 | 80.37678921 | ng/l | 117.554144 | ng/l | 15.08361833 | ng/l |
| Sample | Sample 24 | 5.896934664 | ng/l | −7.957815306 | ng/l | 0.736529164 | ng/l |
| Sample | Sample 26 | 0.507801738 | ng/l | −11.44276737 | ng/l | 0.595191848 | ng/l |
| Sample | Sample 28 | 5.284433816 | ng/l | 6.273367219 | ng/l | 1.867303576 | ng/l |
| Sample | Sample 30 | −6.595630594 | ng/l | −21.70381305 | ng/l | <0.353 | ng/l |
| Sample | Sample 32 | 32.72103719 | ng/l | 34.44972074 | ng/l | 4.340752265 | ng/l |
| Sample | Sample 34 | −4.26865235 | ng/l | −20.25184951 | ng/l | <0.353 | ng/l |
| Sample | Sample 38 | 169.4703786 | ng/l | 253.922559 | ng/l | 29.85608139 | ng/l |
| Sample | Sample 40 | 1.732630582 | ng/l | −8.82912606 | ng/l | 2.432661197 | ng/l |
| Sample | Sample 42 | 20.83978209 | ng/l | 6.757506698 | ng/l | 3.775390601 | ng/l |
| Sample | Sample 44 | 94.83595391 | ng/l | 174.9168092 | ng/l | 22.92946054 | ng/l |
| Sample | Sample 46 | 238.9826051 | ng/l | 619.9832375 | ng/l | 40.6012895 | ng/l |
| Sample | Sample 48 | −7.207993978 | ng/l | −21.41349109 | ng/l | <0.353 | ng/l |

TABLE 15

| Elements | | 146 Nd [1] | | 147 Sm [1] | | 153 Eu [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | <3.200 | ng/l | <3.444 | ng/l | 0.886078135 | ng/l |
| SQStd | Standard 5 ppb | <1.747 | ng/l | <1.940 | ng/l | <0.507 | ng/l |
| Sample | white | <1.747 | ng/l | <1.940 | ng/l | <0.507 | ng/l |
| Sample | Sample 2 | 14.03623654 | ng/l | 1.939953545 | ng/l | 0.939248317 | ng/l |
| Sample | Sample 4 | 12.28903145 | ng/l | 4.655925877 | ng/l | 3.474487341 | ng/l |
| Sample | Sample 6 | 32.55702769 | ng/l | 6.207916463 | ng/l | 4.894180192 | ng/l |
| Sample | Sample 8 | 182.140984 | ng/l | 28.3242142 | ng/l | 12.19602987 | ng/l |
| Sample | Sample 10 | 12.63849646 | ng/l | <1.940 | ng/l | 1.953347408 | ng/l |
| Sample | Sample 12 | 9.493799882 | ng/l | 1.939953545 | ng/l | 2.359037099 | ng/l |
| Sample | Sample 14 | 35.35274245 | ng/l | 7.760000659 | ng/l | 2.56187943 | ng/l |
| Sample | Sample 16 | <1.747 | ng/l | <1.940 | ng/l | 0.736479482 | ng/l |
| Sample | Sample 18 | 35.35245987 | ng/l | 8.535834077 | ng/l | 1.649118145 | ng/l |
| Sample | Sample 36 | 1.806074994 | ng/l | <1.940 | ng/l | 0.330808069 | ng/l |
| Sample | Sample 20 | 726.2750263 | ng/l | 101.2735212 | ng/l | 31.97322833 | ng/l |
| Sample | Sample 22 | 52.47676254 | ng/l | 17.07196711 | ng/l | 2.866004832 | ng/l |
| Sample | Sample 24 | 1.456650304 | ng/l | <1.940 | ng/l | −0.277631987 | ng/l |
| Sample | Sample 26 | 4.252111636 | ng/l | <1.940 | ng/l | 0.026584995 | ng/l |
| Sample | Sample 28 | 8.794864529 | ng/l | 3.879930399 | ng/l | 0.83785481 | ng/l |
| Sample | Sample 30 | <1.747 | ng/l | <1.940 | ng/l | 0.026578902 | ng/l |
| Sample | Sample 32 | 25.21871567 | ng/l | 3.879953709 | ng/l | 1.54773102 | ng/l |
| Sample | Sample 34 | <1.747 | ng/l | <1.940 | ng/l | −0.277638079 | ng/l |
| Sample | Sample 38 | 98.25721473 | ng/l | 15.13180637 | ng/l | 6.719684581 | ng/l |
| Sample | Sample 40 | 4.950982009 | ng/l | <1.940 | ng/l | 1.243483383 | ng/l |
| Sample | Sample 42 | 12.63847647 | ng/l | 5.431874365 | ng/l | 1.344913639 | ng/l |
| Sample | Sample 44 | 110.4901616 | ng/l | 24.05590348 | ng/l | 6.922501382 | ng/l |
| Sample | Sample 46 | 145.7898198 | ng/l | 27.93594969 | ng/l | 5.908261102 | ng/l |
| Sample | Sample 48 | 2.504966527 | ng/l | <1.940 | ng/l | 1.040671804 | ng/l |

TABLE 16

| Elements | | 157 Gd [1] | | 159 Tb [1] | | 163 Dy [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | <2.088 | ng/l | <0.355 | ng/l | <1.575 | ng/l |
| SQStd | Standard 5 ppb | 75.06479713 | ng/l | 2.123966944 | ng/l | <0.947 | ng/l |
| Sample | white | <1.214 | ng/l | <0.210 | ng/l | <0.947 | ng/l |
| Sample | Sample 2 | 5.82980589 | ng/l | 1.242781104 | ng/l | 7.702391566 | ng/l |
| Sample | Sample 4 | 5.343902099 | ng/l | 13.83267285 | ng/l | 2.967958497 | ng/l |
| Sample | Sample 6 | 6.558463524 | ng/l | 4.012324162 | ng/l | 5.429848639 | ng/l |
| Sample | Sample 8 | 20.89033884 | ng/l | 2.963243292 | ng/l | 13.38376586 | ng/l |
| Sample | Sample 10 | 7.530155286 | ng/l | 1.410597383 | ng/l | 6.755461538 | ng/l |
| Sample | Sample 12 | 1.943204929 | ng/l | 0.487443307 | ng/l | 0.695450243 | ng/l |
| Sample | Sample 14 | 9.473345158 | ng/l | 1.620437278 | ng/l | 8.649219018 | ng/l |
| Sample | Sample 16 | 1.943219522 | ng/l | 0.151762732 | ng/l | <0.947 | ng/l |
| Sample | Sample 18 | 8.501698798 | ng/l | 1.410597223 | ng/l | 4.482941726 | ng/l |
| Sample | Sample 36 | 1.700304226 | ng/l | <0.210 | ng/l | 0.884820212 | ng/l |
| Sample | Sample 20 | 96.93097504 | ng/l | 10.81067309 | ng/l | 67.74066042 | ng/l |
| Sample | Sample 22 | 10.93094117 | ng/l | 1.410599944 | ng/l | 10.16442004 | ng/l |
| Sample | Sample 24 | 1.700319051 | ng/l | 0.151755129 | ng/l | 1.074212935 | ng/l |
| Sample | Sample 26 | 6.072721418 | ng/l | 1.452565138 | ng/l | 1.642357336 | ng/l |
| Sample | Sample 28 | 3.886498345 | ng/l | 0.865104603 | ng/l | 1.642300088 | ng/l |
| Sample | Sample 30 | <1.214 | ng/l | 1.410602185 | ng/l | <0.947 | ng/l |
| Sample | Sample 32 | 2.914866346 | ng/l | 7.411407552 | ng/l | 3.536057207 | ng/l |
| Sample | Sample 34 | <1.214 | ng/l | <0.210 | ng/l | 0.695450243 | ng/l |
| Sample | Sample 38 | 17.00366074 | ng/l | 2.837351872 | ng/l | 12.43681344 | ng/l |
| Sample | Sample 40 | <1.214 | ng/l | 0.319599218 | ng/l | <0.947 | ng/l |
| Sample | Sample 42 | 3.643597642 | ng/l | 0.739223426 | ng/l | 2.210467243 | ng/l |
| Sample | Sample 44 | 22.83376313 | ng/l | 4.138240551 | ng/l | 20.58002261 | ng/l |
| Sample | Sample 46 | 34.97997316 | ng/l | 4.13818517 | ng/l | 38.1935795 | ng/l |
| Sample | Sample 48 | 1.214561888 | ng/l | 0.235678434 | ng/l | <0.947 | ng/l |

TABLE 17

| Elements | | 165 Ho [1] | | 166 Er [1] | | 169 Tm [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | <0.348 | ng/l | <1.090 | ng/l | <0.302 | ng/l |
| SQStd | Standard 5 ppb | <0.213 | ng/l | <0.678 | ng/l | <0.191 | ng/l |
| Sample | white | <0.213 | ng/l | <0.678 | ng/l | <0.191 | ng/l |
| Sample | Sample 2 | 0.979848589 | ng/l | 4.882097932 | ng/l | 0.420014016 | ng/l |
| Sample | Sample 4 | 5.154976697 | ng/l | 2.57666833 | ng/l | 0.610928631 | ng/l |
| Sample | Sample 6 | 2.300521405 | ng/l | 3.932812047 | ng/l | 0.458194143 | ng/l |
| Sample | Sample 8 | 1.576269089 | ng/l | 8.272581506 | ng/l | 0.87821282 | ng/l |
| Sample | Sample 10 | 1.405874932 | ng/l | 6.780776783 | ng/l | 1.069143747 | ng/l |
| Sample | Sample 12 | 0.426021306 | ng/l | 1.627349337 | ng/l | 0.190914614 | ng/l |
| Sample | Sample 14 | 1.874500018 | ng/l | 6.916338726 | ng/l | 0.534561387 | ng/l |
| Sample | Sample 16 | <0.213 | ng/l | <0.678 | ng/l | <0.191 | ng/l |
| Sample | Sample 18 | 1.022439287 | ng/l | 3.119170356 | ng/l | 0.267284189 | ng/l |
| Sample | Sample 36 | <0.213 | ng/l | 0.813682881 | ng/l | <0.191 | ng/l |
| Sample | Sample 20 | 10.5233489 | ng/l | 30.92175084 | ng/l | 3.474745725 | ng/l |
| Sample | Sample 22 | 1.19288764 | ng/l | 4.610892312 | ng/l | 0.572753165 | ng/l |
| Sample | Sample 24 | 0.213008053 | ng/l | 1.762976978 | ng/l | <0.191 | ng/l |
| Sample | Sample 26 | 0.383420207 | ng/l | 4.746536506 | ng/l | 0.343649103 | ng/l |
| Sample | Sample 28 | 0.852042612 | ng/l | 2.441056984 | ng/l | 0.649120409 | ng/l |
| Sample | Sample 30 | 0.340813908 | ng/l | <0.678 | ng/l | <0.191 | ng/l |
| Sample | Sample 32 | 0.93724749 | ng/l | 2.034223982 | ng/l | 0.190916945 | ng/l |
| Sample | Sample 34 | <0.213 | ng/l | <0.678 | ng/l | <0.191 | ng/l |
| Sample | Sample 38 | 3.365647025 | ng/l | 8.136962142 | ng/l | 1.183695778 | ng/l |
| Sample | Sample 40 | 0.340811349 | ng/l | 0.67805537 | ng/l | <0.191 | ng/l |
| Sample | Sample 42 | 0.468617204 | ng/l | 0.813674733 | ng/l | 0.305466646 | ng/l |
| Sample | Sample 44 | 5.53851334 | ng/l | 17.08806258 | ng/l | 3.245646323 | ng/l |
| Sample | Sample 46 | 8.478267357 | ng/l | 31.32888206 | ng/l | 3.360210006 | ng/l |
| Sample | Sample 48 | 0.894638591 | ng/l | 0.678071859 | ng/l | <0.191 | ng/l |

TABLE 18

| Elements | | 172 Yb [1] | | 175 Lu [1] | | 178 Hf [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | <1.379 | ng/l | <0.523 | ng/l | <1.995 | ng/l |
| SQStd | Standard 5 ppb | <0.889 | ng/l | <0.343 | ng/l | <1.333 | ng/l |
| Sample | white | <0.889 | ng/l | <0.343 | ng/l | <1.333 | ng/l |
| Sample | Sample 2 | 7.819437704 | ng/l | 1.303336967 | ng/l | 13.20221852 | ng/l |
| Sample | Sample 4 | 4.620598024 | ng/l | 1.097562806 | ng/l | 22.00353903 | ng/l |
| Sample | Sample 6 | 4.087434578 | ng/l | 0.617368603 | ng/l | 2.267910251 | ng/l |
| Sample | Sample 8 | 3.198839679 | ng/l | 0.480181675 | ng/l | 1.201130002 | ng/l |
| Sample | Sample 10 | 5.686848994 | ng/l | 0.75456819 | ng/l | 8.1351519 | ng/l |
| Sample | Sample 12 | 1.777146581 | ng/l | 0.480173269 | ng/l | 1.201130129 | ng/l |
| Sample | Sample 14 | 5.864526758 | ng/l | 1.097554432 | ng/l | 44.94037098 | ng/l |
| Sample | Sample 16 | <0.889 | ng/l | <0.343 | ng/l | <1.333 | ng/l |
| Sample | Sample 18 | 2.132523632 | ng/l | 0.617372855 | ng/l | 2.001223391 | ng/l |
| Sample | Sample 36 | <0.889 | ng/l | <0.343 | ng/l | <1.333 | ng/l |
| Sample | Sample 20 | 22.0377587 | ng/l | 3.018314364 | ng/l | 10.0020586 | ng/l |
| Sample | Sample 22 | 2.13258871 | ng/l | 1.371953474 | ng/l | <1.333 | ng/l |
| Sample | Sample 24 | 2.132567017 | ng/l | 0.960363285 | ng/l | 0.934426865 | ng/l |
| Sample | Sample 26 | <0.889 | ng/l | <0.343 | ng/l | <1.333 | ng/l |
| Sample | Sample 28 | 4.620554639 | ng/l | 0.617364417 | ng/l | 16.40263989 | ng/l |
| Sample | Sample 30 | <0.889 | ng/l | 0.411581882 | ng/l | <1.333 | ng/l |
| Sample | Sample 32 | 1.954845868 | ng/l | 0.480198454 | ng/l | 0.934459673 | ng/l |
| Sample | Sample 34 | <0.889 | ng/l | <0.343 | ng/l | 0.934426865 | ng/l |
| Sample | Sample 38 | 4.975996937 | ng/l | 1.646339956 | ng/l | 19.33649139 | ng/l |
| Sample | Sample 40 | <0.889 | ng/l | 0.342986308 | ng/l | <1.333 | ng/l |
| Sample | Sample 42 | 2.488031007 | ng/l | 0.411581882 | ng/l | 1.467817116 | ng/l |
| Sample | Sample 44 | 24.17005456 | ng/l | 5.487843204 | ng/l | 8.401872331 | ng/l |
| Sample | Sample 46 | 35.01230552 | ng/l | 7.683035539 | ng/l | 74.54825237 | ng/l |
| Sample | Sample 48 | <0.889 | ng/l | <0.343 | ng/l | <1.333 | ng/l |

TABLE 19

| Elements | | 181 Ta [1] | | 182 W [1] | | 195 Pt [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | <0.402 | ng/l | <1.493 | ng/l | <1.902 | ng/l |
| SQStd | Standard 5 ppb | <0.274 | ng/l | <1.036 | ng/l | <1.432 | ng/l |
| Sample | white | <0.274 | ng/l | <1.036 | ng/l | <1.432 | ng/l |
| Sample | Sample 2 | 4.167012321 | ng/l | 7.669885843 | ng/l | 3.436557301 | ng/l |
| Sample | Sample 4 | 0.493451377 | ng/l | 21.35182639 | ng/l | 2.004659723 | ng/l |
| Sample | Sample 6 | <0.274 | ng/l | 4.56045655 | ng/l | 1.431879554 | ng/l |
| Sample | Sample 8 | 0.657941862 | ng/l | 99.09996832 | ng/l | <1.432 | ng/l |
| Sample | Sample 10 | 0.383794383 | ng/l | 4.560417804 | ng/l | 1.718287526 | ng/l |
| Sample | Sample 12 | <0.274 | ng/l | 8.29175399 | ng/l | <1.432 | ng/l |
| Sample | Sample 14 | 11.24042383 | ng/l | 10.77927797 | ng/l | <1.432 | ng/l |
| Sample | Sample 16 | <0.274 | ng/l | 5.389652824 | ng/l | <1.432 | ng/l |
| Sample | Sample 18 | 0.548278175 | ng/l | 176.2442658 | ng/l | <1.432 | ng/l |
| Sample | Sample 36 | <0.274 | ng/l | 6.011660137 | ng/l | <1.432 | ng/l |
| Sample | Sample 20 | 2.083462241 | ng/l | 521.7501266 | ng/l | <1.432 | ng/l |
| Sample | Sample 22 | <0.274 | ng/l | 112.7851477 | ng/l | <1.432 | ng/l |
| Sample | Sample 24 | 0.274140787 | ng/l | 1.865617486 | ng/l | <1.432 | ng/l |
| Sample | Sample 26 | <0.274 | ng/l | 4.767745932 | ng/l | 1.718269775 | ng/l |
| Sample | Sample 28 | 0.383790984 | ng/l | 9.535618379 | ng/l | <1.432 | ng/l |
| Sample | Sample 30 | <0.274 | ng/l | 2.694813365 | ng/l | <1.432 | ng/l |
| Sample | Sample 32 | 0.438624579 | ng/l | 58.046891 | ng/l | <1.432 | ng/l |
| Sample | Sample 34 | <0.274 | ng/l | 13.47424474 | ng/l | <1.432 | ng/l |
| Sample | Sample 38 | 0.822422308 | ng/l | 516.5537286 | ng/l | <1.432 | ng/l |
| Sample | Sample 40 | <0.274 | ng/l | 7.255345824 | ng/l | <1.432 | ng/l |
| Sample | Sample 42 | <0.274 | ng/l | 6.426150341 | ng/l | <1.432 | ng/l |
| Sample | Sample 44 | <0.274 | ng/l | 18.03494325 | ng/l | <1.432 | ng/l |
| Sample | Sample 46 | 5.976387001 | ng/l | 280.7908609 | ng/l | <1.432 | ng/l |
| Sample | Sample 48 | <0.274 | ng/l | 3.109430677 | ng/l | 2.004641971 | ng/l |

TABLE 20

| Elements | | 202 Hg [1] | | 205 Tl [1] | | 208 Pb [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 6.440626972 | ng/l | 12.70382025 | ng/l | 278.0357348 | ng/l |
| SQStd | Standard 5 ppb | <3.614 | ng/l | 4.98729618 | ug/l | −244.3405587 | ng/l |
| Sample | white | <3.614 | ng/l | 1.812229019 | ng/l | −26.20245904 | ng/l |
| Sample | Sample 2 | 4.40261991 | ng/l | 52.05749616 | ng/l | 346.5114798 | ng/l |
| Sample | Sample 4 | <3.614 | ng/l | −3.466681081 | ng/l | 415.1843317 | ng/l |
| Sample | Sample 6 | <3.614 | ng/l | −4.434412956 | ng/l | 1.717503711 | ug/l |
| Sample | Sample 8 | 1.51119265 | ng/l | 7.882586681 | ng/l | 1.778491673 | ug/l |
| Sample | Sample 10 | 1.511147153 | ng/l | 4.187399208 | ng/l | 4.139036493 | ug/l |
| Sample | Sample 12 | <3.614 | ng/l | 66.57966343 | ng/l | 3.854226848 | ug/l |
| Sample | Sample 14 | −2.103373717 | ng/l | 24.68887768 | ng/l | 866.4082441 | ng/l |
| Sample | Sample 16 | −2.103328219 | ng/l | 1.108279665 | ng/l | 848.856003 | ng/l |
| Sample | Sample 18 | 1.511057536 | ng/l | 7.882566548 | ng/l | 1.746336334 | ug/l |
| Sample | Sample 36 | −0.65759253 | ng/l | 17.38518284 | ng/l | 1.266115032 | ug/l |
| Sample | Sample 20 | 18.13751327 | ng/l | 15.88902016 | ng/l | 1.625102184 | ug/l |
| Sample | Sample 22 | −2.826196409 | ng/l | 37.9758031 | ng/l | 686.9998042 | ng/l |
| Sample | Sample 24 | <3.614 | ng/l | 1.372229969 | ng/l | 510.4188 | ng/l |
| Sample | Sample 26 | 5.12571352 | ng/l | −7.865407513 | ng/l | 3.851881586 | ug/l |
| Sample | Sample 28 | 0.06527566 | ng/l | 8.762747194 | ng/l | 587.0826103 | ng/l |
| Sample | Sample 30 | −2.826241906 | ng/l | 11.93026627 | ng/l | 241.4035645 | ng/l |
| Sample | Sample 32 | <3.614 | ng/l | 0.756190226 | ng/l | 222.2911248 | ng/l |
| Sample | Sample 34 | −2.103328564 | ng/l | 2.779913915 | ng/l | 1.314368818 | ug/l |
| Sample | Sample 38 | 15.96936575 | ng/l | −0.211359103 | ng/l | 2.573129 | ug/l |
| Sample | Sample 40 | 9.462965397 | ng/l | 1.196083238 | ng/l | 564.5771625 | ng/l |
| Sample | Sample 42 | <3.614 | ng/l | −3.554585322 | ng/l | 749.9521795 | ng/l |
| Sample | Sample 44 | 1.511056847 | ng/l | 14.39350177 | ng/l | 2.08184032 | ug/l |
| Sample | Sample 46 | 8.017095282 | ng/l | 21.16873763 | ng/l | 8.014462702 | ug/l |
| Sample | Sample 48 | <3.614 | ng/l | 12.01769804 | ng/l | 4.075080774 | ug/l |

TABLE 21

| Elements | | 209 Bi [1] | | 232 Th [1] | | 238 U [1] | |
|---|---|---|---|---|---|---|---|
| | Sample | | Sequence | | Sequence | | Sequence |
| Type | name | Conc. | unit | Conc. | unit | Conc. | unit |
| SQBlk | White HNO3 | 2.302596768 | ng/l | <0.795 | ng/l | <0.519 | ng/l |
| SQStd | Standard 5 ppb | <0.462 | ng/l | <0.639 | ng/l | 1.58671578 | ng/l |
| Sample | white | 2.599086068 | ng/l | <0.639 | ng/l | <0.417 | ng/l |
| Sample | Sample 2 | 12.49544483 | ng/l | <0.639 | ng/l | 15.43560737 | ng/l |
| Sample | Sample 4 | 0.009467276 | ng/l | 1.663631876 | ng/l | 11.597759 | ng/l |
| Sample | Sample 6 | −1.285269623 | ng/l | 2.43019345 | ng/l | 14.93491336 | ng/l |
| Sample | Sample 8 | 0.194494602 | ng/l | 12.65202541 | ng/l | 7.927019915 | ng/l |
| Sample | Sample 10 | 29.69987812 | ng/l | 6.90216674 | ng/l | 50.23111091 | ng/l |
| Sample | Sample 12 | −0.730368093 | ng/l | <0.639 | ng/l | 2.337515752 | ng/l |
| Sample | Sample 14 | 18.78522948 | ng/l | <0.639 | ng/l | 41.63517511 | ng/l |
| Sample | Sample 16 | −0.175460918 | ng/l | <0.639 | ng/l | 0.251976609 | ng/l |
| Sample | Sample 18 | 12.86550371 | ng/l | 13.80194392 | ng/l | 30.95475423 | ng/l |
| Sample | Sample 36 | −1.840182707 | ng/l | <0.639 | ng/l | 0.41881113 | ng/l |
| Sample | Sample 20 | 4.171377714 | ng/l | 47.15486294 | ng/l | 69.42651209 | ng/l |
| Sample | Sample 22 | 0.009502202 | ng/l | 2.813515785 | ng/l | 25.61489383 | ng/l |
| Sample | Sample 24 | 0.749419239 | ng/l | <0.639 | ng/l | 0.835921058 | ng/l |
| Sample | Sample 26 | −0.175478204 | ng/l | 0.38590879 | ng/l | 7.509820092 | ng/l |
| Sample | Sample 28 | 1.026811708 | ng/l | 1.15256857 | ng/l | 12.26553685 | ng/l |
| Sample | Sample 30 | <0.462 | ng/l | <0.639 | ng/l | <0.417 | ng/l |
| Sample | Sample 32 | −1.470250294 | ng/l | 3.963407249 | ng/l | 24.86368098 | ng/l |
| Sample | Sample 34 | 4.448782177 | ng/l | 1.024737313 | ng/l | 5.340721368 | ng/l |
| Sample | Sample 38 | 3.33900875 | ng/l | 31.56390402 | ng/l | 182.4878291 | ng/l |
| Sample | Sample 40 | 0.841871518 | ng/l | 5.879966047 | ng/l | 10.17973693 | ng/l |
| Sample | Sample 42 | −1.562737763 | ng/l | 1.535808539 | ng/l | 10.59661154 | ng/l |
| Sample | Sample 44 | 2.044150142 | ng/l | 1.280276886 | ng/l | 25.5311508 | ng/l |
| Sample | Sample 46 | 7.130961197 | ng/l | 21.34114849 | ng/l | 99.56245533 | ng/l |
| Sample | Sample 48 | <0.462 | ng/l | <0.639 | ng/l | 2.504350114 | ng/l |

In order to check the relevance of the measurements, dilution tests were performed on a wine sample (ref. Wine No. 20) at 3 levels: twentyfold, tenfold and fivefold dilution in nitric acid.

The semi-quantitative analysis was performed on the main elements. For each element, the obtained measurements were compared (fivefold dilution value/tenfold dilution value) and (tenfold dilution value/twentyfold dilution value). When, for these two values, the ratio is equal to 2+−0.5, the measurement of the element can be considered to be reliable in the field and the element can be considered to be reliable to use. Thus, 50 reliable elements were found to be measured for this sample: Na, Mg, Al, S. K, Ca, Sc, V, Cr, Mn, Fe, Co, Ni, Cu, Ge, Rb, Sr, Y, Zr, Mo, Pd, I, Cs, Ba, La, Ce, Pr, Nd, Eu, Gd, Tb, Dy. Tm, Yb, W, TI, Pb, Bi, Th and U.

When a value of the ratio is equal to 2+−0.5 and the other value is equal to 2+−1, the element can be measured more specifically by adjusting the dilution setting, and can be considered to be worthwhile understanding. In this case, 11 additional elements have been found for this sample: Si, Ti, Zn, Ga, Br, Nb, In, Sn, Sm, Ho, Er.

Then, finally, there are 2 elements, the two ratios of which are equal to 2+−1: Cd, HI.

It has been found that more than 60 mineral elements thus can be measured by ICP-MS semi-quantitative analysis on this sample and entered into a metallic profile file of the sample. See tables 22 to 40 below.

TABLE 22
Sample

| | | | Data | | | |
|---|---|---|---|---|---|---|
| Line | | Data | acquisition | | Sample | Vial |
| No. | | file | time | Type | name | No |
| 3 | La[1]: The ratio of the analyte to the hybrid ion interference | DATA01.D | Nov. 25, 2021 | StdSQ | White | 1001 |
| | source = 0.20% is below the permitted minimum = 1.00% | | 3:20 PM | | HNO3 | |
| | Cr[1]: The ratio of the analyte to the oxide ion interference | | | | | |
| | source = 0.02% is below the permitted minimum = 1.00% | | | | | |
| | V[1]: The ratio of analyte to the ion oxide interference | | | | | |
| | source = 0.03% is below the permitted minimum = 1.00%. | | | | | |
| 4 | La[1]: The ratio of the analyte to the hybrid ion interference | DATA02.D | Nov. 25, 2021 | StdSQ | Standard | 1002 |
| | source = 0.22% is below the permitted minimum = 1.00% | | 3:26 PM | | 5 ppb | |
| | Pd[1]: The ratio of the analyte to the oxide ion interference | | | | | |
| | source = 0.17% is below the permitted minimum = 1.00% | | | | | |
| | Zr[1]: The ratio of the analyte to the oxide ion interference | | | | | |
| | source = 0.07% is below the permitted minimum = 1.00% | | | | | |
| | Cr[1]: The ratio of the analyte to the oxide ion interference | | | | | |
| | source = 0.02% is below the permitted minimum = 1.00% | | | | | |
| | V[1]: The ratio of the analyte to the oxide ion interference | | | | | |
| | source = 0.02% is below the permitted minimum = 1.00% | | | | | |
| 5 | La[1]: The ratio of the analyte to the hybrid ion interference | DATA03.D | Nov. 25, 2021 | Sample | white | 1001 |
| | source = 0.35% is below the permitted minimum = 1.00% | | 3:32 PM | | | |
| | Cr[1]: The ratio of the analyte to the oxide ion interference | | | | | |
| | source = 0.02% is below the permitted minimum = 1.00% | | | | | |
| | V[1]: The ratio of the analyte to the oxide ion interference | | | | | |
| | source = 0.02% is below the permitted minimum = 1.00% | | | | | |
| 6 | Hf[1]: The ratio of the analyte to the argide ion interference | DATA04.D | Nov. 25, 2021 | Sample | Sample 20 | 1003 |
| | source = 0.06% is below the permitted minimum = 0.10% | | 3:39 PM | | diluted 20x | |
| | Ti[1]: The ratio of the analyte to the oxide ion interference | | | | | |
| | source = 0.83% is below the permitted minimum = 1.00% | | | | | |
| 7 | Re[1]: The ratio of the analyte to the hybrid ion interference | DATA05.D | Nov. 25, 2021 | Sample | Sample | 1004 |
| | source = 0.26% is below the permitted minimum = 1.00% | | 3:45 PM | | 20 diluted | |
| | Hf[1]: The ratio of the analyte to the argide ion interference | | | | 10 x | |
| | source = 0.08% is below the permitted minimum = 0.10% | | | | | |
| | Br[1]: The ratio of the analyte to the argide ion interference | | | | | |
| | source = 0.00% is below the permitted minimum = 0.10% | | | | | |
| | Ti[1]: The ratio of the analyte to the oxide ion interference | | | | | |
| | source = 0.57% is below the permitted minimum = 1.00% | | | | | |
| 8 | Hg[1]: The ratio of the analyte to the oxide ion interference | DATA06.D | Nov. 25, 2021 | Sample | Sample | 1005 |
| | source = 0.51% is below the permitted minimum = 1.00% | | 3:51 PM | | 20 diluted | |
| | Re[1]: The ratio of the analyte to the hybrid ion interference | | | | 5 x | |
| | source = 0.11% is below the permitted minimum = 1.00% | | | | | |
| | Hf[1]: The ratio of the analyte to the argide ion interference | | | | | |
| | source = 0.10% is below the permitted minimum = 0.10% | | | | | |
| | Ti[1]: The ratio of the analyte to the oxide ion interference | | | | | |
| | source = 0.44% is below the permitted minimum = 1.00% | | | | | |
| 9 | Cr[1]: The ratio of the analyte to the oxide ion interference | DATA07.D | Nov. 25, 2021 | Sample | white | 1001 |
| | source = 0.03% is below the permitted minimum = 1.00% | | 3:57 PM | | | |
| | V[1]: The ratio of the analyte to the oxide ion interference | | | | | |
| | source = 0.01% is below the permitted minimum = 1.00% | | | | | |
| | Mg[1]: The ratio of the analyte to the hybrid ion interference | | | | | |
| | source = 0.67% is below the permitted minimum = 1.00% | | | | | |

TABLE 23

| Sample | | | | | | | | | | | | | |
|---|---|---|---|---|---|---|---|---|---|---|---|---|---|
| | | Data | | | | 7 Li [1] | | 9 Be [1] | | 11 B [1] | | 23 Na [1] | |
| Line | Data | acquisition | | Sample | Vial | | SQ | | SQ | | SQ | | SQ |
| No | file | time | Type | name | No. | Conc. | Unit | Conc. | Unit | Conc. | Unit | Conc | Unit |
| Z | Z | Z | Z | Z | Z | Z | Z | Z | Z | Z | Z | Z | Z |

| | | | | | | | | |
|---|---|---|---|---|---|---|---|---|
| 3 | | ug/l | <321.170 | ng/l | 920.0862 | ng/l | 6.948212 | ug/l |
| 4 | 5 | ug/l | <213.033 | ng/l | 661.5147 | ng/l | 91.81326 | ug/l |
| 5 | <163.385 | ng/l | <213.033 | ng/l | 661.5187 | ng/l | 81.8914 | ug/l |
| 6 | <163.385 | ng/l | <213.033 | ng/l | 223.2624 | ug/l | 1.779179 | mg/l |
| 7 | 555.5447 | ng/l | <213.033 | ng/l | 551.6463 | ug/l | 3.983916 | mg/l |
| 8 | 424.814 | ng/l | <213.033 | ng/l | 1.458855 | mg/l | 9.302541 | mg/l |
| 9 | <163.385 | ng/l | <213.033 | ng/l | 1.617062 | ug/l | 77.26582 | ug/l |

TABLE 24

| Sample | | | | | | | | | | | | | |
|---|---|---|---|---|---|---|---|---|---|---|---|---|---|
| | | Data | | | | 24 Mg [1] | | 27 Al [1] | | 28 Si [1] | | 31 P [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |
| Z | Z | Z | Z | Z | Z | Z | Z | Z | Z | Z | Z | Z | Z |

| | | | | | | | | |
|---|---|---|---|---|---|---|---|---|
| 3 | 5 | ug/l | 674.9172 | ng/l | 149.3038 | ug/l | 23.67625 | ug/l |
| 4 | 5 | ug/l | 5.411928 | ug/l | 792.1571 | ug/l | 93.83786 | ug/l |
| 5 | 71.65856 | ug/l | 6.765013 | ug/l | 1.360774 | mg/l | 176.7563 | ug/l |
| 6 | 23.2304 | mg/l | 201.4889 | ug/l | 6.113642 | mg/l | 56.24164 | mg/l |
| 7 | 53.4616 | mg/l | 443.9274 | ug/l | 13.40428 | mg/l | 188.1198 | mg/l |
| 8 | 129.8615 | mg/l | 1.058415 | mg/l | 38.73764 | mg/l | 615.3672 | mg/l |
| 9 | 1.297309 | ug/l | 368.981 | ng/l | 557.3863 | ug/l | 44.98991 | ug/l |

TABLE 25

| Sample | | | | | | | | | | | | | |
|---|---|---|---|---|---|---|---|---|---|---|---|---|---|
| | | Data | | | | 34 S [1] | | 35 Cl [1] | | 39 K [1] | | 42 Ca [1] | |
| Line | Data | acquisition | | Sample | Vial | | SQ | | SQ | | SQ | | SQ |
| No. | file | time | Type | name | No. | Conc. | Unit | Conc. | Unit | Conc: | Unit | Conc. | Unit |
| Z | Z | Z | Z | Z | Z | Z | Z | Z | Z | Z | Z | Z | Z |

| | | | | | | | | |
|---|---|---|---|---|---|---|---|---|
| 3 | 1.330119 | mg/l | 51.37264 | mg/l | 46.31177 | ug/l | 274.5236 | ug/l |
| 4 | 4.294925 | mg/l | 274.6534 | mg/l | 69.39287 | ug/l | 322.417 | ug/l |
| 5 | 4.43233 | mg/l | 255.906 | mg/l | 66.5062 | ug/l | 316.652 | ug/l |
| 6 | 18.24528 | mg/l | 256.6122 | mg/l | 51.33366 | mg/l | 1.019905 | mg/l |
| 7 | 37.66809 | mg/l | 283.908 | mg/l | 120.9462 | mg/l | 1.963175 | mg/l |
| 8 | 90.00942 | mg/l | 312.2381 | mg/l | 285.6995 | mg/l | 4.239254 | mg/l |
| 9 | 823.8419 | ug/l | 225.6763 | mg/l | 39.88207 | ug/l | 72.20595 | ug/l |

TABLE 26

| Sample | | | | | | | | | | | | | |
|---|---|---|---|---|---|---|---|---|---|---|---|---|---|
| | | Data | | | | 45 Sc [1] | | 47 Ti [1] | | 51 V [1] | | 52 Cr [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |
| Z | Z | Z | Z | Z | Z | Z | Z | Z | Z | Z | Z | Z | Z |

| | | | | | | | | |
|---|---|---|---|---|---|---|---|---|
| 3 | 71.68516 | ng/l | <147.440 | ng/l | 15.35417 | ng/l | 64.02564 | ng/l |
| 4 | 48.07574 | ng/l | 139.6728 | ng/l | 8.924951 | ng/l | 23.7962 | ng/l |
| 5 | 70.43703 | ng/l | <99.759 | ng/l | 12.74999 | ng/l | 22.4443 | ng/l |
| 6 | 111.8065 | ng/l | 14.42854 | ug/l | 12.80881 | ug/l | 1.57226 | ug/l |
| 7 | 169.9468 | ng/l | 33.23656 | ug/l | 29.94204 | ug/l | 3.630541 | ug/l |
| 8 | 392.4629 | ng/l | 84.7986 | ug/l | 74.51491 | ug/l | 8.497346 | ug/l |
| 9 | 10.06221 | ng/l | <99.759 | ng/l | 4.249957 | ng/l | 30.82714 | ng/l |

TABLE 27

| Sample | | | | | | | | | | | | | |
|---|---|---|---|---|---|---|---|---|---|---|---|---|---|
| | | Data | | | | 55 Mn [1] | | 56 Fe [1] | | 59 Co [1] | | 60 Ni [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | 1.309176 | ug/l | 26.61071 | ug/l | 5 | ug/l | 126.4132 | ug/l |
| 4 | 181.5448 | ng/l | 563.322 | ng/l | 5 | ug/l | 195.5752 | ng/l |
| 5 | 263.4466 | ng/l | 2.550708 | ug/l | 5.401359 | ng/l | 143.9955 | ng/l |
| 6 | 30.25458 | ug/l | 86.6288 | ug/l | 103.1015 | ng/l | 1.20895 | ug/l |
| 7 | 71.26405 | ug/l | 195.2384 | ug/l | 237.6841 | ng/l | 2.708845 | ug/l |
| 8 | 167.2919 | ug/l | 467.7948 | ug/l | 534.5359 | ng/l | 5.937916 | ug/l |
| 9 | 42.87059 | ng/l | 75.73899 | ng/l | <0.563 | ng/l | 12.78926 | ng/l |

TABLE 28

| Sample | | | | | | | | | | | | | |
|---|---|---|---|---|---|---|---|---|---|---|---|---|---|
| | | Data | | | | 63 Cu [1] | | 66 Zn [1] | | 69 Ga [1] | | 72 Ge [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | 117.0231 | ug/l | 5.022928 | mg/l | 51.98229 | ug/l | 23.39162 | ug/l |
| 4 | 350.0294 | ng/l | 871.4449 | ng/l | 75.48039 | ng/l | 14.13688 | ng/l |
| 5 | 111.5177 | ng/l | 4.112336 | ug/l | 51.1206 | ng/l | 10.60271 | ng/l |
| 6 | 1.511884 | ug/l | 16.13005 | ug/l | 557.7521 | ng/l | 77.75516 | ng/l |
| 7 | 3.373097 | ug/l | 37.50732 | ug/l | 1.305454 | ug/l | 132.5369 | ng/l |
| 8 | 8.001922 | ug/l | 96.74405 | ug/l | 3.615819 | ug/l | 300.4266 | ng/l |
| 9 | 5.875441 | ng/l | 302.1156 | ng/l | <1.715 | ng/l | <8.835 | ng/l |

TABLE 29

| Sample | | | | | | | | | | | | | |
|---|---|---|---|---|---|---|---|---|---|---|---|---|---|
| | | Data | | | | 75 As [1] | | 79 Br [1] | | 85 Rb [1] | | 88 Sr [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | <20.871 | ug/l | 4.117618 | mg/l | 21.31329 | ug/l | 220.7309 | ug/l |
| 4 | <10.622 | ng/l | 1.49998 | ug/l | 6.743935 | ng/l | 41.30447 | ng/l |
| 5 | <10.622 | ng/l | 1.331993 | ug/l | 2.247935 | ng/l | 12.36767 | ng/l |
| 6 | 1.49173 | ug/l | 3.216096 | ug/l | 30.31787 | ug/l | 4.853062 | ug/l |
| 7 | 4.477012 | ug/l | 3.936183 | ug/l | 63.5898 | ug/l | 10.77329 | ug/l |
| 8 | 13.93315 | ug/l | 7.87313 | ug/l | 147.3391 | ug/l | 24.8568 | ug/l |
| 9 | 14.87171 | ng/l | 431.9915 | ng/l | 20.87405 | ng/l | <1.167 | ng/l |

TABLE 30

| Sample | | | | | | 89 Y [1] | | 90 Zr [1] | | 93 Nb [1] | | 95 Mo [1] | |
|---|---|---|---|---|---|---|---|---|---|---|---|---|---|
| Line | Data | Data acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | <12.499 | ug/l | 90.44702 | ug/l | <11.023 | ug/l | 71.97346 | ug/l |
| 4 | 5 | ug/l | 6.751991 | ng/l | <0.525 | ng/l | <2.249 | ng/l |
| 5 | <0.536 | ng/l | 4.705898 | ng/l | <0.525 | ng/l | 3.148796 | ng/l |
| 6 | 105.6551 | ng/l | 198.7269 | ng/l | 106.8144 | ng/l | 452.663 | ng/l |
| 7 | 256.3127 | ng/l | 473.3603 | ng/l | 274.6744 | ng/l | 1.081231 | ug/l |
| 8 | 552.8305 | ng/l | 1.061635 | ug/l | 646.9966 | ng/l | 2.508672 | ug/l |
| 9 | <0.536 | ng/l | <1.023 | ng/l | <0.525 | ng/l | 2.698949 | ng/l |

TABLE 31

| Sample | | | | | | | | | | | | | |
|---|---|---|---|---|---|---|---|---|---|---|---|---|---|
| | | Data | | | | 101 Ru [1] | | 103 Rh [1] | | 105 Pd [1] | | 107 Ag [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | <19.972 | ug/l | <3.092 | ug/l | <14.780 | ug/l | 9.666594 | ug/l |
| 4 | <1.093 | ng/l | <0.176 | ng/l | 13.69562 | ng/l | 1.014247 | ng/l |
| 5 | <1.093 | ng/l | <0.176 | ng/l | <0.878 | ng/l | 0.894923 | ng/l |
| 6 | <1.093 | ng/l | <0.176 | ng/l | 2.98486 | ng/l | 1.2529 | ng/l |
| 7 | <1.093 | ng/l | <0.176 | ng/l | 4.56512 | ng/l | 1.252893 | ng/l |
| 8 | <1.093 | ng/l | 0.388115 | ng/l | 10.00823 | ng/l | 1.312574 | ng/l |
| 9 | <1.093 | ng/l | <0.176 | ng/l | <0.878 | ng/l | <0.298 | ng/l |

TABLE 32

| Sample | | | | | | | | | | | | | |
|---|---|---|---|---|---|---|---|---|---|---|---|---|---|
| | | Data | | | | 111 Cd [1] | | 115 In [1] | | 118 Sn [1] | | 121 Sb [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | <38.448 | ug/l | <6.357 | ug/l | 33.89751 | ug/l | <29.980 | ug/l |
| 4 | <2.462 | ng/l | <0.422 | ng/l | 19.27826 | ng/l | <2.126 | ng/l |
| 5 | <2.462 | ng/l | <0.422 | ng/l | 2.326593 | ng/l | 4.252896 | ng/l |
| 6 | 7.878688 | ng/l | 0.421761 | ng/l | 69.47017 | ng/l | 17.43684 | ng/l |
| 7 | 12.31045 | ng/l | 0.927863 | ng/l | 180.8423 | ng/l | 39.12731 | ng/l |
| 8 | 34.46979 | ng/l | 2.446237 | ng/l | 412.2904 | ng/l | 122.0658 | ng/l |
| 9 | <2.462 | ng/l | <0.422 | ng/l | <1.662 | ng/l | <2.126 | ng/l |

TABLE 33

| Sample | | | | | | | | | | | | | |
|---|---|---|---|---|---|---|---|---|---|---|---|---|---|
| | | Data | | | | 125 Te [1] | | 127 I [1] | | 133 Cs [1] | | 137 Ba [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | <595.327 | ug/l | 378.3952 | ug/l | <6.739 | ug/l | 2.979674 | mg/l |
| 4 | <43.579 | ng/l | 15.73595 | ng/l | <0.539 | ng/l | 493.1233 | ng/l |
| 5 | <43.579 | ng/l | 21.56411 | ng/l | <0.539 | ng/l | 234.5314 | ng/l |
| 6 | <43.579 | ng/l | 135.2189 | ng/l | 280.8072 | ng/l | 2.307008 | ug/l |
| 7 | <43.579 | ng/l | 320.5936 | ng/l | 652.0469 | ng/l | 4.41259 | ug/l |
| 8 | <43.579 | ng/l | 781.854 | ng/l | 1.442961 | ug/l | 9.652219 | ug/l |
| 9 | <43.579 | ng/l | 5.245201 | ng/l | <0.539 | ng/l | <3.077 | ng/l |

TABLE 34

| Sample | | | | | | | | | | | | | |
|---|---|---|---|---|---|---|---|---|---|---|---|---|---|
| | | Data | | | | 139 La [1] | | 140 Ce [1] | | 141 Pr [1] | | 146 Nd [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | 3.596801 | ug/l | 5 | ug/l | <2.089 | ug/l | <10.657 | ug/l |
| 4 | 0.668293 | ng/l | 5 | ug/l | 0.355145 | ng/l | <0.887 | ng/l |
| 5 | 0.546777 | ng/l | 0.722352 | ng/l | <0.178 | ng/l | <0.887 | ng/l |
| 6 | 305.0768 | ng/l | 400.6317 | ng/l | 46.96845 | ng/l | 158.6919 | ng/l |
| 7 | 684.6781 | ng/l | 896.8478 | ng/l | 100.1643 | ng/l | 340.7452 | ng/l |
| 8 | 1.549269 | ug/l | 2.049287 | ug/l | 225.586 | ng/l | 779.8986 | ng/l |
| 9 | <0.304 | ng/l | <0.241 | ng/l | <0.178 | ng/l | <0.887 | ng/l |

TABLE 35

| Sample | | | | | | | | | | | | | |
|---|---|---|---|---|---|---|---|---|---|---|---|---|---|
| | | Data | | | | 147 Sm [1] | | 153 Eu [1] | | 157 Gd [1] | | 159 Tb [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | <12.680 | ug/l | <3.443 | ug/l | <8.593 | ug/l | <1.552 | ug/l |
| 4 | <1.007 | ng/l | <0.266 | ng/l | 31.28687 | ng/l | 0.769256 | ng/l |
| 5 | <1.007 | ng/l | <0.266 | ng/l | 0.904922 | ng/l | <0.113 | ng/l |
| 6 | 22.5696 | ng/l | 6.716031 | ng/l | 23.27047 | ng/l | 2.669849 | ng/l |
| 7 | 57.63607 | ng/l | 16.04464 | ng/l | 56.24096 | ng/l | 6.222394 | ng/l |
| 8 | 122.5381 | ng/l | 36.51763 | ng/l | 138.4955 | ng/l | 14.73191 | ng/l |
| 9 | <1.007 | ng/l | <0.266 | ng/l | <0.646 | ng/l | <0.113 | ng/l |

TABLE 36

| Sample | | | | | | | | | | | | | |
|---|---|---|---|---|---|---|---|---|---|---|---|---|---|
| | | Data | | | | 163 Dy [1] | | 165 Ho [1] | | 166 Er [1] | | 169 Tm [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | <7.346 | ug/l | <1.741 | ug/l | <5.866 | ug/l | <1.758 | ug/l |
| 4 | <0.518 | ng/l | <0.118 | ng/l | <0.382 | ng/l | <0.109 | ng/l |
| 5 | <0.518 | ng/l | <0.118 | ng/l | <0.382 | ng/l | <0.109 | ng/l |
| 6 | 14.69972 | ng/l | 2.008018 | ng/l | 5.344032 | ng/l | 0.851684 | ng/l |
| 7 | 35.92392 | ng/l | 4.84315 | ng/l | 15.6508 | ng/l | 1.790762 | ng/l |
| 8 | 84.48959 | ng/l | 13.08947 | ng/l | 34.28205 | ng/l | 3.51599 | ng/l |
| 9 | <0.518 | ng/l | <0.118 | ng/l | <0.382 | ng/l | <0.109 | ng/l |

TABLE 37

| Sample | | | | | | | | | | | | | |
|---|---|---|---|---|---|---|---|---|---|---|---|---|---|
| | | Data | | | | 172 Yb [1] | | 175 Lu [1] | | 178 Hf [1] | | 181 Ta [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | <8.769 | ug/l | <3.656 | ug/l | <15.506 | ug/l | <3.519 | ug/l |
| 4 | <0.517 | ng/l | <0.203 | ng/l | <0.804 | ng/l | <0.169 | ng/l |
| 5 | <0.517 | ng/l | <0.203 | ng/l | 0.964825 | ng/l | <0.169 | ng/l |
| 6 | 5.684221 | ng/l | 0.730789 | ng/l | 2.412046 | ng/l | 0.573153 | ng/l |
| 7 | 11.1619 | ng/l | 2.841997 | ng/l | 6.271295 | ng/l | 0.708016 | ng/l |
| 8 | 27.28581 | ng/l | 4.100652 | ng/l | 15.91973 | ng/l | 2.798377 | ng/l |
| 9 | <0.517 | ng/l | <0.203 | ng/l | <0.804 | ng/l | <0.169 | ng/l |

TABLE 38

| Sample | | | | | | | | | | | | | |
|---|---|---|---|---|---|---|---|---|---|---|---|---|---|
| | | Data | | | | 182 W [1] | | 185 Re [1] | | 189 Os [1] | | 193 Ir [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | <14.917 | ug/l | <13.663 | ug/l | <44.267 | ug/l | 13.24563 | ug/l |
| 4 | <0.651 | ng/l | <0.533 | ng/l | <1.502 | ng/l | <0.314 | ng/l |
| 5 | <0.651 | ng/l | <0.533 | ng/l | <1.502 | ng/l | 0.31358 | ng/l |
| 6 | 138.3977 | ng/l | <0.533 | ng/l | <1.502 | ng/l | <0.314 | ng/l |
| 7 | 328.1097 | ng/l | 0.745729 | ng/l | <1.502 | ng/l | <0.314 | ng/l |
| 8 | 733.2534 | ng/l | 0.745729 | ng/l | <1.502 | ng/l | <0.314 | ng/l |
| 9 | <0.651 | ng/l | <0.533 | ng/l | <1.502 | ng/l | <0.314 | ng/l |

TABLE 39

| Sample | | | | | | | | | | | | | |
|---|---|---|---|---|---|---|---|---|---|---|---|---|---|
| | | Data | | | | 195 Pt [1] | | 197 Au [1] | | 202 Hg [1] | | 205 Tl [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | <44.357 | ug/l | 66.2043 | ug/l | <321.779 | ug/l | | ug/l |
| 4 | <0.991 | ng/l | 2.419613 | ng/l | 2.65644 | ng/l | 5 | ug/l |
| 5 | <0.991 | ng/l | 1.209758 | ng/l | <2.656 | ng/l | 0.334272 | ng/l |
| 6 | 1.189543 | ng/l | 1.382644 | ng/l | 14.87621 | ng/l | 7.688404 | ng/l |
| 7 | <0.991 | ng/l | <0.864 | ng/l | 13.81362 | ng/l | 19.05529 | ng/l |
| 8 | <0.991 | ng/l | <0.864 | ng/l | 16.47022 | ng/l | 46.87466 | ng/l |
| 9 | 1.387784 | ng/l | <0.864 | ng/l | <2.656 | ng/l | <0.334 | ng/l |

TABLE 40

| Sample | | | | | | | | | | | | | |
|---|---|---|---|---|---|---|---|---|---|---|---|---|---|
| | | Data | | | | 208 Pb [1] | | 209 Bi [1] | | 232 Th [1] | | 238 U [1] | |
| Line | Data | acquisition | | Sample | Vial | | SQ | | SQ | | SQ | | SQ |
| No. | file | time | Type | name | No. | Conc. | Unit | Conc. | Unit | Conc. | Unit | Conc. | Unit |

| | | | | | | | | |
|---|---|---|---|---|---|---|---|---|
| 3 | ######## | mg/l | ######## | mg/l | | ug/l | | ug/l |
| 4 | 24.99437 | ng/l | 1.124546 | ng/l | <0.485 | ng/l | 2.535931 | ng/l |
| 5 | 16.52643 | ng/l | <0.351 | ng/l | <0.485 | ng/l | 0.380387 | ng/l |
| 6 | 621.9238 | ng/l | 2.389696 | ng/l | 17.47886 | ng/l | 20.92329 | ng/l |
| 7 | 1.339312 | ug/l | 4.287383 | ng/l | 35.73659 | ng/l | 53.64927 | ng/l |
| 8 | 2.959643 | ug/l | 9.559019 | ng/l | 85.56777 | ng/l | 118.4951 | ng/l |
| 9 | <0.510 | ng/l | <0.351 | ng/l | <0.485 | ng/l | <0.317 | ng/l |

[Step (e)] Generating the Database

For each wine, the measured metallic compositions are gathered and shown in a digital table (Excel in these examples).

The additional data concerning the wine are gathered and also entered on the table. In particular, the names and descriptions of the wines, the data relating to the origins, the data relating to the additional physico-chemical analyses, and, finally, the qualitative data relating to the taste and the quality of the wine, or the sensations via the scores and the sales prices.

The following parameters, as long as they are available, are consolidated and recorded in the table for each collected wine:

Origin/AOC/AOP/IGP/Wine—growing name—Other Names (variety, cuvée, etc.)/Type/Vintage/Cultivation Mode/% Alcohol/Selling Price/Sulfites/Internet Site of the Domain and Contact Domain/Guard Time/Country/Region/Wine-growing region/GPS Coordinates/Appellation/Type of Glass Container (in ml)/Stopper/Storage/Date of opening/Site of Opening/View/Nose/Mouth/Climate/Exposure-Precise Orientation/Influence of the Wind/Relief/Altitude/Watering source/Irrigation of the vine/Type of soil/Cultivation types/Surface area in Ha/Planting density/Fertilization of the vine/Green covering/Plant-health pruning age control/Type of of the vine/Average of the vine/Type of fermentation/Picking/Sorting/Destemming/Types of presses/Maceration time of the skins and seeds in the must/Wine tank material/Wine tank volume (in hl)/Addition of yeast/Bonding & Clarification/Filtration/Blending/Critical Scores: Oenologist/Public Experts (Vivino, Robert Parker (Wine Advocate), Wine Spectator, Decanting, Jancis Robinson, James Suckling, JEB DUNNUCK, Vinous, Hachette, Wine Enthusiast)/Label/Certifications/Medals.

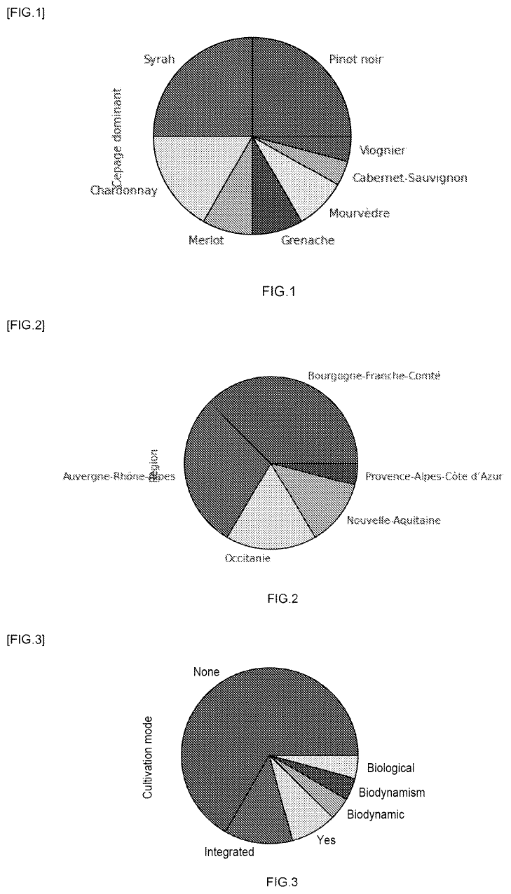

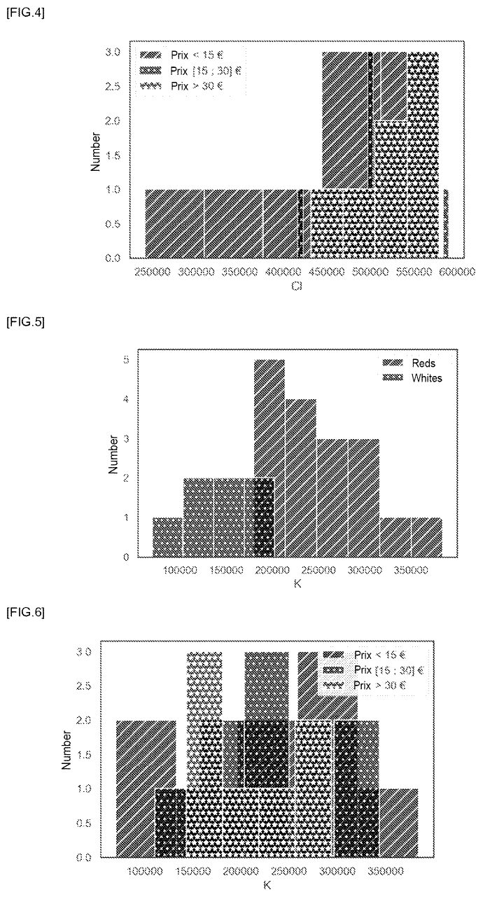

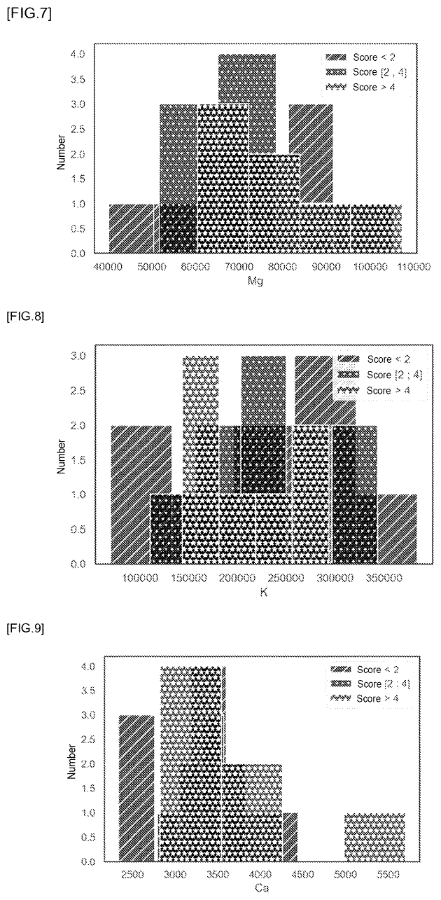

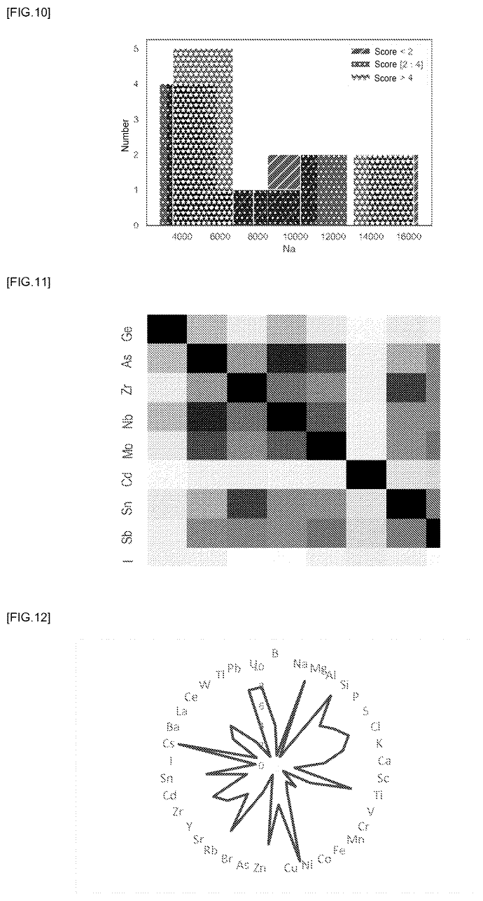

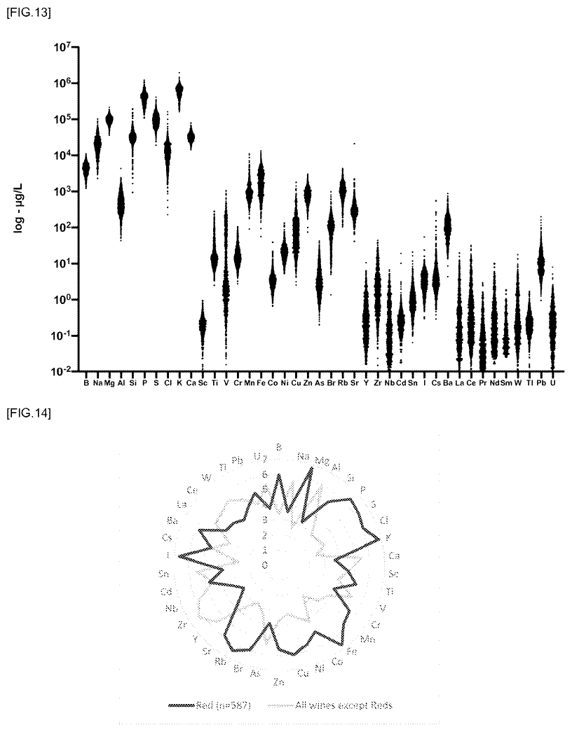

For the analyzed wines, appended FIGS. 1 to 3 show, by way of examples, the distribution that is obtained as a function, respectively, of the information concerning the grape varieties, the origins, regions, and the cultivation modes.

[Step (h)] Preliminary Statistical Studies

In order to analyze the data, in this example, Python language is used with the NumPy, Pandas, Seaborn and Matplotlib libraries (non-exhaustive list). The working method begins with initial data processing by analyzing the basic properties of all the data. Next, the phase of viewing certain features according to previously defined classes (for example, consolidation by price ranges, score ranges, etc.).

Loading Data into Python

The following libraries are imported into Python: Pandas, Seaborn (for the graphical representation), NumPy, Matplotlib and Scipy.stats. The Excel files are loaded in the form of DataFrame Pandas: the first contains the concentrations of metals, the second contains information relating to the origins, the descriptions and the quality of the wine. These files are merged in order to have only one DataFrame, consolidated by virtue of the common information: “Sample Name”.

### Creation of a DataFrame Subset

Seaborn is used to create histograms of the quantitative variables with the seaborn.histplot function. The Seaborn KDE option is superposed on the histogram. This is a representation of the data using the probability density curve.

Seaborn.plot.pie is then implemented in order to represent the qualitative variables such as the origin, the type of wine, the cultivation mode, etc.

In order to better graphically represent the data, subsets of interest are created. Examples of studied subsets are as follows: the metals per category, the prices (by setting lower and upper limits) and the Vivino scores.

These subsets are included in new DataFrame Pandas.
Representations of quantitative variables
   Therefore, it is now possible to trace histograms of the metals for each subset, namely, the histograms of the concentrations of the metals belonging to the price subsets, then the type of wine and finally the Vivino score.

Once a relationship is identified between two variables (for example, the price and the Vivino score), it is possible to use Seaborn.Implot to visually confirm the relationship.

### Correlations

In order to quantitatively establish a correlation, the “.corr” function is used in the following format: DataFrame.corr( ). A correlation of 0.75 is thus obtained between the price and the Vivino score, which proves that the variables are correlated. This item of information is found by means of the “seaborn.clustermap” graph, which consolidates the most correlated metals into clusters. This graph is symmetrical, and the correlation values on the diagonal are equal to 1, since it is the correlation between the metal and itself. By plotting this graph for “ultra-trace” type metals, significant correlations are observed between the metals La, Ce, Pr, Nd, and Eu.

### Additional Analyses

Finally, a graph used herein but which is relatively overloaded is the seaborn.pairplot graph. On the abscissa it shows the variables x₁, . . . , xₙ as a function of the ordinates of the variables x₁, . . . , xₙ₋₁. In this case, this involves the main metals. The diagonal simply shows the histogram of the metal xₙ. It is then possible to visually search for correlations.

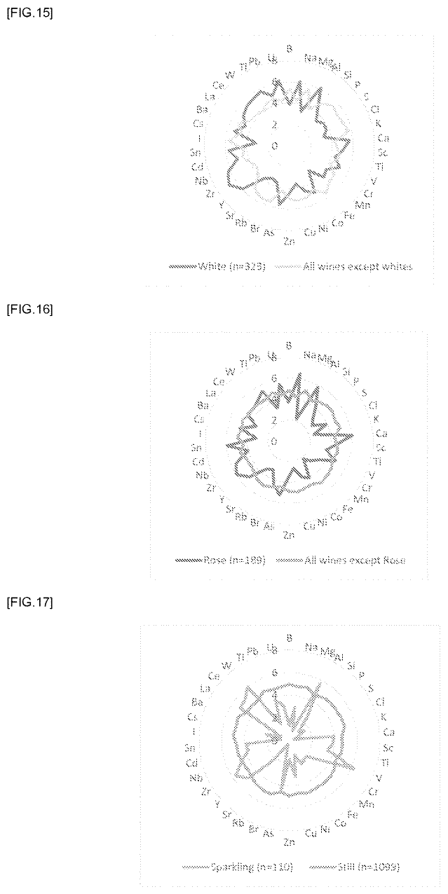

FIGS. 4, 5 and 6 are examples of graphs that are obtained, in which:
the ordinate corresponds to the number of wines entering the category (price for FIGS. 4 and 5 and red or white wine for FIG. 6) and counted; and
the abscissa corresponds to the concentration (in CI for FIG. 4 and in K for FIGS. 5 and 6) expressed in μg/L.

The graph of FIG. 4 shows the chlorine content, which increases with the price of the wine.

The graph of FIG. 6 shows a difference between white and red wine in terms of the potassium content.

The 4 graphs of FIGS. 7 to 10 show the distribution of the concentrations of Mg, K, Ca and Na, respectively, as a function of the quality of the wine (in this case expressed by a selection of the obtained scores). A profile can be distinguished that is plotted for quality wines, with scores of more than 4, which have preferential contents for the 4 elements Mg. K. Ca and Na centered, respectively, around 650,000, 200,000, 3,250 and 5,000 g/L.

[Step (i)] Use of the Statistically Analyzed Data

Based on the various measurements performed on the ultra-traces, a correlation matrix is obtained, as shown in FIG. 11.

The scale on the right-hand side of FIG. 11 corresponds to the Pearson coefficient (the value-1 indicates that the variables have inversely correlated, +1 indicates linear correlation and zero indicates no correlation). It is computed according to the covariance expression of the matrix (X, Y) divided by std(X)*std(Y) (standard deviation of X times standard deviation of Y): cov(X, Y)/std(X)std(Y). The Pearson coefficient is an index reflecting a linear relationship between two continuous variables. The correlation coefficient varies between −1 and +1, with 0 reflecting a zero relationship between the two variables, with a negative value (negative correlation) meaning that when one of the variables increases, the other decreases; while a positive value (positive correlation) indicates that the two variables vary together in the same direction.

A very clear correlation is seen, for example, in terms of the lanthanides between them, and is also fairly strongly associated with W, As and Nb.

### 2^{nd} Series of Examples: Paragraphs to [0221] to [0226]

The samples are taken from a commercial wine bottle, namely AOC Saint Joseph Domaine, Le Pizon 2020, Fabrice and Loic Peychon.

The protocol described for the first series of examples in paragraphs to is implemented, except that, during sampling, the wine is stored after threefold dilution in 1% ultra-pure nitric acid (HNO₃), with the addition of an internal indium standard at 10 μg/gL. The use of an acidic solution such as HNO₃ allows the wine to be stabilized while limiting the problems of mineral precipitation and adsorption on the walls of the containers. In addition, the use of an internal standard notably allows any evaporation to be controlled during storage. The samples are again diluted fivefold in 1% HNO₃ immediately before the analyses. These analyses are performed semi-quantitatively and in helium collision mode.

Table 41 below shows the results that were obtained:

TABLE 41

| Dec. 19, 2022 | Dilution | 7 Li [1] | 9 Be [1] | 11 B [1] | 23 Na [1] | 24 Mg [1] | 27 Al [1] | 28 Si [1] | 31 P [1] |
|---|---|---|---|---|---|---|---|---|---|
| 3:45 PM | 1S | <10.865 | <7.999 | 4323.117951 | 6420.225248 | 134744.4057 | 87.68768508 | 46856.64378 | 475222.3338 |

| 34 S [1] | 35 Cl [1] | 39 K [1] | 42 Ca [1] | 45 Sc [1] | 47 Ti [1] | 51 V [1] | 52 Cr [1] |
|---|---|---|---|---|---|---|---|
| 126766.5793 | 21125.17991 | 794795.976 | 29496.90574 | 0.06970004 | 31.34875312 | 0.874501194 | 5.202111907 |
| 55 Mn [1] | 56 Fe [1] | 59 Co [1] | 60 Ni [1] | 63 Cu [1] | 66 Zn [1] | 69 Ga [1] | 72 Ge [1] |
| 753.8040735 | 1633.823306 | 2.48942815 | 111.9944544 | 72.52318772 | 1261.691735 | 10.00928184 | 0.337436474 |

| 75 As [1] | 78 Se [1] | 79 Br [1] | 85 Rb [1] | 88 Sr [1] | 89 Y [1] | 90 Zr [1] | 93 Nb [1] |
|---|---|---|---|---|---|---|---|
| 0.652684469 | −5.266672295 | 92.97613738 | 1800.528751 | 306.1536213 | 0.146846262 | 0.953228557 | 0.02609793 |

| 95 Mo [1] | 101 Ru [1] | 103 Rh [1] | 105 Pd [1] | 107 Ag [1] | 111 Cd [1] | 118 Sn [1] | 121 Sb [1] |
|---|---|---|---|---|---|---|---|
| −0.176680211 | <0.045 | <0.008 | <0.050 | −0.086705733 | <0.131 | 0.903406934 | −2.160388289 |

| 125 Te [1] | 127 I [1] | 133 Cs [1] | 137 Ba [1] | 139 La [1] | 140 Ce [1] | 141 Pr [1] | 146 Nd [1] |
|---|---|---|---|---|---|---|---|
| <1.165 | 2.619138237 | 13.04405149 | 71.20891766 | 0.107973696 | 0.193258774 | 0.00608641 | <0.038 |

| 147 Sm [1] | 153 Eu [1] | 157 Gd [1] | 159 Tb [1] | 163 Dy [1] | 165 Ho [1] | 166 Er [1] | 169 Tm [1] |
|---|---|---|---|---|---|---|---|
| <0.042 | <0.011 | <0.027 | 0.003687661 | <0.021 | 0.007546048 | <0.015 | <0.004 |

| 172 Yb [1] | 175 Lu [1] | 178 Hf [1] | 181 Ta [1] | 182 W [1] | 185 Re [1] | 189 Os [1] | 193 Ir [1] |
|---|---|---|---|---|---|---|---|
| <0.020 | 0.021636942 | <0.050 | <0.006 | 0.061637559 | <0.019 | <0.053 | <0.011 |

| 195 Pt [1] | 197 Au [1] | 202 Hg [1] | 205 Tl [1] | 208 Pb [1] | 209 Bi [1] | 232 Th [1] | 238 U [1] |
|---|---|---|---|---|---|---|---|
| <0.034 | <0.029 | −0.102958381 | 0.071520107 | 20.11692756 | −1.178604435 | 0.023340202 | 0.274785129 |

FIG. 12 shows the mineral profile of the analyzed wine, in the form of a Kiviat diagram that shows the results in deciles and with reference to all the 590 red wines of France analyzed under the same conditions.

Note concerning FIG. 12: a specific profile of the wine is shown, showing, for example, relative to all the red wines, a very high relative concentration of Ni and Cs (last deciles) and a high magnesium and silicon content (before the last decile); however, the wine is very sodium and aluminum poor.

3^{rd} series of examples: paragraphs to [0227] to [230]

The Samples of 1,200 Commercial Wines Produced in France, with Various Natures, Regions, domains, AOCs and vintages, are sampled and analyzed in the same way as for the second series of examples.

The analyses conducted within the context of this study of 1,200 wines produced in France perfectly illustrate the distribution of the metallic and mineral elements over the various classes of concentrations (FIG. 13).

The results of analyses performed on these 1,200 wines yield mineral profiles depicted using Kiviat diagrams in FIGS. 14 to 17, in which:
FIG. 14 shows a mineral profile (dark curve) associated with the average of n=587 red wines compared to a mineral profile (clear curve) associated with the average of (1,200-587) non-red wines;
FIG. 15 shows a mineral profile (dark curve) associated with the average of n=323 white wines, compared to a mineral profile (clear curve) associated with the average of (1,200-323) non-white wines;
FIG. 16 shows a mineral profile (dark curve) associated with the average of n=189 rosé wines compared to a mineral profile (clear curve) associated with the average of (1,200-189) non-rosé wines;
FIG. 17 shows a mineral profile (dark curve) associated with the average of n=110 sparkling wines compared to a mineral profile (clear curve) associated with the average of (1,200-110) non-sparkling wines.

These profiles allow a genuine mineral signature to be assigned to the wines. Clearly, on average representations of the categories of red, white, rosé and sparkling wines (FIGS. 14 to 17, respectively), there is a clear differentiation of their mineral compositions compared to those of the other wines.

1. A method, notably a computer-implemented method, for managing and/or monitoring at least one factor *f^{x}* selected from a set of factors comprising:
*f¹* the agricultural production of a raw material for producing an alcoholic beverage, preferably wine;
*f²* the production of this alcoholic beverage;
*f³* the storage of this alcoholic beverage;
*f⁴* the maturation of this alcoholic beverage;
*f⁵* the consumption of this alcoholic beverage;
*f⁶* the quality of this alcoholic beverage;
*f⁷* the authenticity of this alcoholic beverage relative to a reference selected from the group comprising, advantageously formed by: the names of the wine and the domains, the appellations of origin; the geographical indications; the traditional specialties guaranteed; the labels; the trademarks; and the combinations thereof;
*f⁸* the traceability of this alcoholic beverage;
*f⁹* the selling price of this alcoholic beverage;
said method mainly involving:
(a) collecting at least one, preferably at least two, samples of the alcoholic beverage, placing each of them in an inert container relative to the alcoholic beverage and sealably closing said container with an equally inert stopper;
(b) assigning data to each sample relating to the alcoholic beverage, which data is selected from the group comprising, advantageously formed by: data relating to the origin, data relating to the vineyard site, data relating to production, data relating to storage and maturation, data relating to consumption, physico-chemical data, qualitative data, in particular organoleptic data, economic data, commercial data, and combinations of these data;
(c) optionally storing at least some of the samples collected in step (a) under determined conditions;
(d) analyzing each sample in order to determine at least one mineral profile, preferably a metallic profile;
(e) forming a database relating to the samples and derived from step (b) and step (d);
(f) optionally, completing and/or updating the data assigned in step (b), at least once, over all or some of the samples;
(g) optionally, completing and/or repeating the analyses performed in step (d), at least once, over all or some of the samples derived from step (c);
(h) processing these data by means of a statistical analysis, advantageously by means of automatic or semi-automatic methods based on statistical processing, and even more advantageously by “datamining”, preferably using artificial intelligence tools and/or other “datamining” techniques; and
(i) using the processed data for managing and/or monitoring at least one of the aforementioned factors *f^{x}*.

2. The method as claimed in claim 1, wherein the number N of alcoholic beverages collected in step (a) is such that, in an ascending order of preference:
N≥500; N≥1,000; N≥10,000.

3. The method as claimed in claim 1, wherein:
the data relating to the origin includes the name of the alcoholic beverage, the name of the producer, the name of the domain, the year of production, the sample collection date, the name of the cuvée, the batch number, and/or the type of alcoholic beverage;
the data relating to the vineyard site includes the appellation of origin, the geographical indication, the country, the region, the site, the plot, the grape varieties, the distribution of the grape varieties, the exposure, the sunshine, the planting density (in feet/ha), the type of pruning of the vine, the cultivation mode, the fertilization of the vine, the green cover, the plant-health control, the watering, the average age of the vine, the relief, the type of soil, the source of the water, and/or the irrigation of the vine;
the data relating to production includes the type of grape harvest, the date of the grape harvests, the type of sorting and destemming, the type of vinification, the type of press, the maceration time of the skins and seeds in the must, the material of the tanks, whether or not yeast is added, the type of bonding and clarification, the filtration system, and/or the blending;
the data relating to storage includes the storage time, the successive container types, the container, the temperature, the humidity, the stopper type, and/or the packaging date;
the data relating to consumption includes the presence and the content of sulfites, the presence and the content of phenolic compounds, the percentage of alcohol, and/or the presence and the content of aromatic compounds;
the qualitative data, in particular organoleptic data, includes assessments of the Balance, Length, Intensity, Complexity and Type [BLIC(T) method], the color, the flavors, tastes, and/or the duration of the impression of the flavors of the wine in the mouth, preferably expressed as cuadalies;
the economic data includes the price, the sales volume, and/or the sales amount;
the commercial data includes the labels, competition awards/medals, classifications, and/or received scores.

4. The method as claimed in claim 1, wherein the mineral profile analyzed in step (d) for the samples comprises:
at least 5 mineral elements (called main elements) selected from B, Na, Mg, P, S, Cl, K, Ca;
at least the following metallic elements (called oligo-metals): Fe; Cu; Zn; Mn;
optionally at least one of the following metallic elements: Pb and Cd;
at least 10, preferably at least 30, and, more preferably, at least 40 elements selected from the following trace mineral elements: Rb, Cs, Sr, Ba, Ce, Ti, V, Cr, Co, Ni, Zr, Mo, Ag, Al, Ga, Sn, As, Br, I, Se;
and/or from the following ultra-trace mineral elements: La, Pr, Nd, Sm, Eu, Gd, Tb, Dy, Ho, Er, Tm, Yb, Lu, Th, U, Sc, Y, Nb, Ru, Rh, Pd, Hf, Ta, W, Re, Os, Ir, Pt, Au, Kg, TI, Bi, Sb;
optionally at least a portion of the isotopes of these elements;
the measurements of the concentrations of these elements; and/or
the ratios of concentrations of these elements, and, optionally, all or some of their isotopes.

5. The method as claimed in claim 1, wherein step (d) comprises analyzing chemical and physical parameters of the alcoholic beverage, with these parameters preferably being selected for an alcoholic beverage formed by wine, in the group advantageously formed by: ABV (Alcohol Strength by Volume), glucose+fructose, TA (Total Acidity), acetic acid, free SO₂, total SO₂, pH, active SO₂, ethanal, malic acid, lactic acid, CO₂, tartaric acid, gluconic acid, glycerol, optical density (absorbance at one or more wavelengths of 280, 420, 520 and 620 nm) and all the combinations of these parameters.

6. The method as claimed in claim 1, wherein processing the data according to step (h) mainly involves:
using at least one of the following means:
exploratory analyses and logistic regressions for completing classifications based on Principal Component Analysis (PCA);
discriminant analyses (or LDA (Linear Discriminant Analysis));
predictive model algorithm, preferably selected from the group comprising, ideally formed by: Random Forest (RF) Decision Forests and/or Artificial Neural Networks (ANN) and/or Support Vector Machines (SVM);
performing at least one of the following actions:
identifying and predicting the origins of the various mineral elements within a wine;
identifying and predicting the impacts on the quality of a wine and the evolution of the quality of a wine of the various mineral elements within a wine;
adjusting the origins in order to adapt the quality.

7. The method as claimed in claim 1 wherein, the data processed in step (h) includes:
at least one mineral analytical profile, advantageously a metallic profile, of the alcoholic beverage, measured in step (d) from the following elements:
at least 5 mineral elements (called main elements) selected from B, Na, Mg, P, S, Cl, K, Ca;
at least the following metallic elements (called oligo-metals): Fe; Cu; Zn; Mn;
optionally at least one of the following metallic elements: Pb and Cd;
at least 10, preferably at least 30 and, more preferably, at least 40 elements selected from the following trace mineral elements: Rb, Cs, Sr, Ba, Ce, Ti, V, Cr, Co, Ni, Zr, Mo, Ag, Al, Ga, Sn, As, Br, I, Se;
and/or from the following ultra-trace mineral elements: La, Pr, Nd, Sm, Eu, Gd, Tb, Dy, Ho, Er, Tm, Yb, Lu, Th, U, Sc, Y, Nb, Ru, Rh, Pd, Hf, Ta, W, Re, Os, Ir, Pt, Au, Kg, TI, Bi, Sb;
optionally at least a portion of the isotopes of these elements;
the measurements of the concentrations of these elements;
and/or the ratios of concentrations of these elements, and, optionally of all or some of their isotopes;
and at least 1, preferably at least 5, physicochemical parameters of the alcoholic beverage selected from the group of parameters comprising, advantageously formed by: ABV (Alcohol Strength by Volume), glucose+fructose, TA (Total Acidity), acetic acid, free SO₂, total SO₂, pH, active SO₂, ethanal, malic acid, lactic acid, CO₂, tartaric acid, gluconic acid, glycerol, optical density, and all the combinations of these parameters.

8. The method as claimed in claim 6, wherein the use of the data processed in step (h) for managing and/or monitoring the quality (factor *f⁶*) of the alcoholic beverage, mainly involves:
identifying one or more mineral profiles, preferably metallic profiles, each forming a specific target signature of a certain level of quality for an alcoholic beverage or an alcoholic beverage promised to be at a certain level of quality;
searching for and selecting from a group of alcoholic beverages, the one or more alcoholic beverages for which the mineral profile, preferably a metallic profile, corresponds to a target signature;
marking this or these selected beverages using an acquired or forthcoming quality assurance label;
optionally using the mineral profiles, preferably metallic profiles, of the non-selected alcoholic beverages in order to anticipate negative evolutions of these beverages and to provide the necessary corrective solutions.

9. The method as claimed in claim 1, wherein the use of the data processed in step (h) for managing and/or monitoring the authenticity (factor *f⁷*) of the alcoholic beverage, mainly involves:
identifying one or more metallic profiles forming specific signatures of the origin of the alcoholic beverage;
using this or these signatures as markers guaranteeing the authenticity of the alcoholic beverage;
detecting counterfeits using these markers.

10. A device for implementing the method as claimed claim 1, comprising a sample library comprising at least one enclosure, which houses and stores the samples collected in step (a) of the method in inert containers each closed by a stopper, and in that this enclosure is able to place these samples under given temperature, pressure, humidity, as well as atmospheric conditions.
